(19) United States
(12) Patent Application Publication
(10) Pub. No.: US 2025/0030211 A1
(43) Pub. Date: Jan. 23, 2025

(54) ELECTRICAL ENERGY CONNECTION ASSEMBLY AND HANDHELD ELECTROSURGICAL TOOL

(71) Applicant: FlexDex, Inc., Brighton, MI (US)

(72) Inventors: Matthew P. Weber, Brighton, MI (US); Zachary R. Zimmerman, Northville, MI (US); James Duncan Geiger, Ottawa Hills, OH (US)

(21) Appl. No.: 18/775,474

(22) Filed: Jul. 17, 2024

Related U.S. Application Data
(60) Provisional application No. 63/527,176, filed on Jul. 17, 2023.

(51) Int. Cl. H01R 39/64; A61B 18/00; A61B 18/14

(52) U.S. Cl. CPC H01R 39/64; A61B 18/1445; A61B 2018/00178; A61B 2018/00595; H01R 2201/12

(57) ABSTRACT

An electrical energy connection assembly is provided that can be equipped in a handheld electrosurgical tool or other medical device, as well as non-medical devices, among many other possible applications. An electrical energy conduction and delivery facilitated via the electrical energy connection is uninterrupted amid rotational movements about a rotational degree of freedom between a pair of bodies or assemblies. Drawbacks encountered in past electrosurgical tool applications such as torque buildup, inhibited instrument maneuverability, visual or physical obstruction, and/or unplanned disconnections, are altogether avoided with use of the electrical energy connection assembly.

### CROSS-REFERENCE TO RELATED APPLICATION

This application claims the benefit of U.S. Provisional Patent Application No. 63/527,176, with a filing date of Jul. 17, 2023, the contents of which are hereby incorporated by reference in their entirety.

### TECHNICAL FIELD

The present disclosure relates generally to electrical energy connection assemblies and to articulating remote access tools and, more particularly, to handheld electrosurgical tools employed for use in minimally invasive surgical (MIS) procedures and remote access surgical procedures.

### BACKGROUND

A monopolar or bipolar electrosurgical device is intended to supply high-frequency electrical current to cut, coagulate, desiccate, and fulgurate tissue. Energy is supplied by an electrosurgical generator via an electrical cord and delivered to a connector on the instrument. Energy is controlled by a trained surgeon via a foot pedal or a device mounted switch. With traditional electrosurgical instruments, the receiving connector on the instrument is typically located at a proximal end of the device handle or at a proximal end of the instrument shaft. The receiving connector is also typically fully constrained, or rigidly connected to the instrument, and therefore moves with motions of the instrument.

When an electrosurgical generator cord is connected to an instrument, it introduces instrument positioning limitations to the user of the device such as a surgeon. For instance, a connected cable may wrap around the instrument if the surgeon twists or rotates the device about the instrument shaft. Consequently, the connected cable may introduce a buildup of torque on the device that the surgeon must exert additional effort to counteract the impact thereof. Moreover, the location of the connected cable may introduce a visual or physical obstruction to use of other surgical devices. Lastly, a rigid connection has potential to disconnect in situations where the cable is snagged during motion of the instrument. Collectively, these drawbacks introduce constraints on the surgeon, limit the range of motion for instrument use, and may dampen the tactile feedback that the surgeons receive through the instrument.

Electrosurgical devices are intended to be used by trained surgeons leading a procedure. But there are occasions where use may be by others assisting the surgeon at the surgeon's direction. Radiofrequency (RF) energy can be activated at the instrument via use of a foot pedal or a device-mounted switch.

### SUMMARY

In an embodiment, an electrical energy connection assembly may include a user input assembly, a first electrically conductive body, one or more electrical connectors, and one or more second electrically conductive bodies. The user input assembly has one or more electrically nonconductive bodies. The first electrically conductive body is constrained translationally to the electrically nonconductive body(ies). The first electrically conductive body has a rotational degree of freedom with respect to the electrically nonconductive body(ies). The rotational degree of freedom is about an axis of rotation of the electrically nonconductive body(ies). The rotational degree of freedom provides full rotational capabilities of the first electrically conductive body and the electrically nonconductive body(ies) relative to each other. The electrical connector(s) is carried by the first electrically conductive body and can be rotated with the first electrically conductive body about the rotational degree of freedom. When the electrical connector(s) is connected to an external electrical energy source, electrical energy is conducted from the electrical connector(s), to the first electrically conductive body, to the second electrically conductive body(ies), and to an output body located remote of the user input assembly.

In another embodiment, an electrical energy connection assembly may include a first electrically conductive body and a second electrically conductive body. The second electrically conductive body is constrained translationally with respect to the first electrically conductive body. The second electrically conductive body has a rotational degree of freedom with respect to the first electrically conductive body. The rotational degree of freedom provides unlimited rotational capabilities of the first electrically conductive body and the second electrically conductive body with respect to each other. When the electrical energy connection assembly is connected to an external electrical energy source, electrical energy is conducted from the first electrically conductive body, to the second electrically conductive body, and to an output body that is located remote of the first electrically conductive body and that is located remote of the second electrically conductive body. Further, the electrical energy conduction remains substantially uninterrupted during unlimited rotation between the first electrically conductive body and the second electrically conductive body.

In another embodiment, a method of delivering electrical energy between electrically conductive bodies may include a multitude of steps. One step may involve providing a translational degree of constraint between a first electrically conductive body and a second electrically conductive body, and providing an unlimited rotational degree of freedom between the first electrically conductive body and the second electrically conductive body. When connected to an external electrical energy source, electrical energy is delivered from the first electrically conductive body and to the second electrically conductive body. Further, the electrical energy delivery between the first and second electrically conductive bodies is substantially uninterrupted during unlimited rotations between the first and second electrically conductive bodies about the unlimited rotational degree of freedom.

Further scope of applicability of the present disclosure will become apparent from the detailed description given hereinafter. But it should be understood that the detailed description and specific examples, while indicating exemplary embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The present disclosure will become more fully understood from the detailed description given below and the accompanying drawings, which are given by way of illustration only, and do not limit the present disclosure, and wherein:

FIG. 1 is a schematic block diagram of an embodiment of an electrical energy connection assembly;

FIG. 2 is a schematic block diagram of an embodiment of an electrical energy connection assembly;

FIG. 3 is a schematic block diagram of an embodiment of an electrical energy connection assembly;

FIG. 4 is a schematic block diagram of an embodiment of an electrical energy connection assembly;

FIG. 5 is a schematic block diagram of an embodiment of an electrical energy connection assembly;

FIG. 6 is a schematic block diagram of an embodiment of an electrical energy connection assembly;

FIG. 7 is a side view of an electrosurgical instrument;

FIG. 8 is a side view of an embodiment of an electrical energy connection assembly implemented in a handheld electrosurgical tool;

FIG. 9 is an enlarged view of the electrical energy connection assembly of FIG. 8;

FIG. 10 is a sectional view of an embodiment of an electrical energy connection assembly;

FIG. 11 is an exploded view of the electrical energy connection assembly of FIG. 10;

FIG. 12 is a sectional view of an embodiment of a shaft and cables;

FIG. 13 is a sectional view of an embodiment of an electrical energy connection assembly;

FIG. 14 is a sectional view of an embodiment of an electrical energy connection assembly;

FIG. 15 is a sectional view of an embodiment of a shaft and cables;

FIG. 16 is a sectional view of an embodiment of an electrical energy connection assembly;

FIG. 17 is a sectional view of an embodiment of an electrical energy connection assembly;

FIG. 18 is a sectional view of an embodiment of an electrical energy connection assembly;

FIG. 19 is a sectional view of an embodiment of an electrical energy connection assembly; and

FIG. 20 is a schematic depiction of an embodiment of an electrical energy connection assembly that facilitates bipolar electrical energy transference.

### DETAILED DESCRIPTION

Multiple embodiments of electrical energy connection assemblies and handheld electrosurgical tools are depicted in the figures and detailed in this description. Definitions of certain technical terms used herein are presented prior to particular figure references in this description:

Body—A body is a discrete continuous component that can be used as structural components to form an assembly or sub-assembly. The displacement/motion state of a body can be completely defined with respect to a reference ground by six degrees of freedom (DoF). A body can be part of an assembly, where the assembly may include multiple bodies that are inter-connected by joints. Generally, a body may be rigid (i.e., with no compliance) or may be compliant. One or more discrete bodies may be connected together via a rigid joint. These bodies together are still termed as a body as there are no single or multi degree of freedom joints between these bodies. In certain scenarios, this body may be produced out of a single/monolithic structure and therefore, be only a single body. In certain scenarios, a body may be compliant (i.e., not rigid) but still discrete and continuous. In any case, the body may be monolithic or assembled using rigid joints. The body may be of homogenous material composition or heterogeneous material composition.

Conductive Body—A body that is conductive will allow for the transfer of electricity between separate and discrete bodies that are maintaining physical contact to one another. A series of conductive bodies that maintain physical contact will create a path for electricity to travel. Although a conductive body may have some degree of internal electrical resistance, it is a passive element and not intended to alter an electrical waveform. Terms namely “conductive body” and “conducting body” may be used interchangeably throughout this patent.

Insulated Body—A body that is insulated will prevent the transfer of electricity within and between bodies. Terms namely “insulated body,” “insulating body,” and “nonconductive body” may be used interchangeably throughout this patent.

Mechanism/Joint/Connector—In general, there may be a certain equivalence between the terms “mechanism” and “joint.” A “joint” may be alternatively referred to as a “connector” or a “constraint.” All of these can be viewed as allowing certain rigid body motion(s) along certain degree(s) of freedom between two bodies and constraining the remaining motions. A mechanism generally includes multiple joints and bodies. Typically, a joint may be of simpler construction, while a mechanism may be more complex as it can include multiple joints. A joint refers to a mechanical connection that allows motions, as opposed to a fixed joint (e.g., welded, bolted, screwed, or glued jointly). In the latter case, fixed joint, two bodies are fused with each other and are considered one-and-the-same in the kinematic sense (i.e., because there is no relative motion allowed or there are no relative degrees of freedom between the two). The term “fixed joint” may be used herein to refer to this kind of joint between two bodies. When reference to the term “joint” is made, it means a connection that allows at least some motions or degrees of freedom (e.g., a pin joint, a pivot joint, a universal joint, a ball and socket joint, etc.).

Electrical Joint—An electrical joint refers to an interface between two bodies that is electrically conductive. Although typically accompanied by a mechanical joint that has some degree of rigidity, an electrical joint in and of itself is not a mechanically structural joint. If a mechanical joint is composed of materials that are electrically conductive, then the mechanical joint may also be an electrical joint. Electrical joints typically have low electrical resistance. Electrical joints may also be electrically insulated from surrounding regions and bodies. When two electrically conductive bodies are joined, an electrical connection is established.

Degree of Freedom (DoF)—As noted, a joint or mechanism allows certain rigid body motions between two bodies and constrains the remaining motions. “Degrees of freedom” is a technical term to capture or convey these allowed “motions.” In total, there are six independent motions and therefore degrees of freedom possible between two rigid bodies when there is no joint between them: three translations and three rotations. A joint will allow anywhere between zero and six DoFs between the two bodies. For the case when the joint allows zero DoFs, this effectively becomes a “fixed joint,” as described above, where the two bodies are rigidly fused or connected to each other. In this case, from a kinematic sense, the two bodies are one-and-the-same. For the case when the joint allows six DoFs, this effectively means that the joint does not constrain any motions between the two bodies. In other words, the motions of the two bodies are entirely independent of each other.

Degree of Constraint (DOC)—“Degree of constraint” refers to directions along which relative motion is constrained between two bodies. Since relative motion is constrained, these are directions along which motion and loads (i.e., forces or moments) can be transmitted from one body to the other body. Since the joint does not allow relative motion between the two bodies in the DoC direction, if one body moves in the DoC direction, it drives along with it the other body along that direction. In other words, motions are transmitted from one rigid body to another in the DoC directions. Consequently, loads are also transmitted from one rigid body to another in the DOC directions, which are sometimes also referred to as the load bearing directions or simply bearing directions. The term “retention” may also be used in the context of a DoC direction. For example, one body may be constrained or equivalently retained with respect to a second body along a certain DoC. This means that relative motion is not allowed between the two bodies in the DoC direction, or equivalently the direction of constraint, or equivalently the direction of retention. Retention of all six DoFs means the same thing as having six DoC between two bodies.

Local Ground—In the context of an assembly of bodies connected by joints (e.g., a multi-body system, a mechanism), one or more bodies may be referred to as the “reference” or “ground” or “local ground.” The body referred to as the local ground is not necessarily an absolute ground (i.e., attached or bolted to the actual ground). Rather, the body that is selected as a local ground simply serves as a mechanical reference with respect to which the motions of all other bodies are described or investigated.

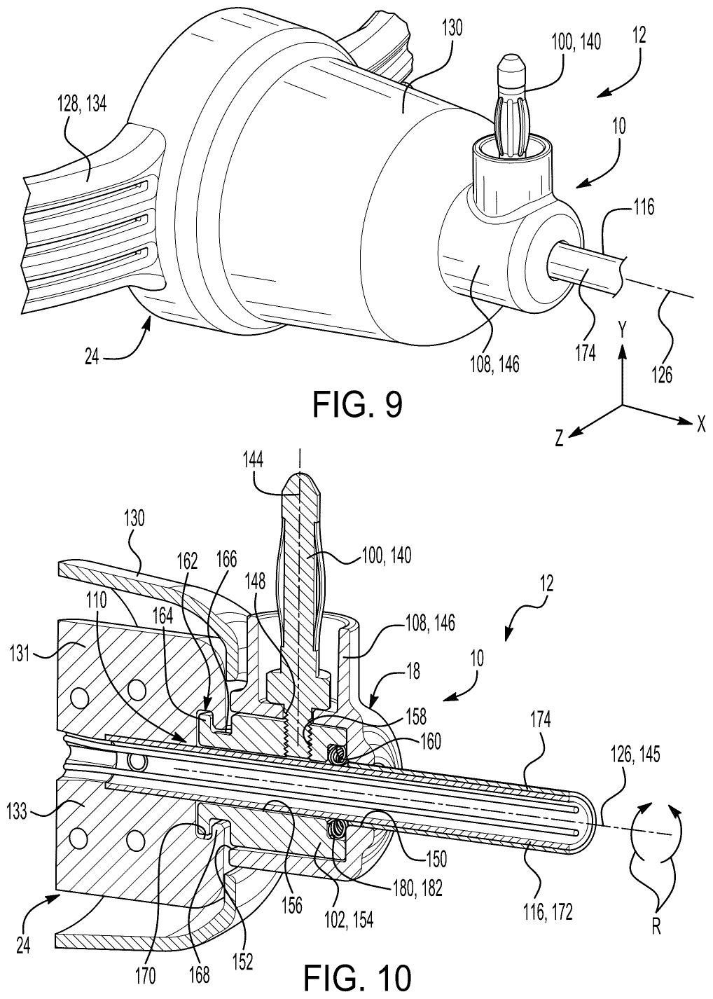

Axis and Direction—Axis refers to a specific line in space. A body may rotate with respect to (w.r.t.) another body about a certain axis. Alternatively, a body may translate w.r.t. another body in a certain direction. A direction is not defined by a particular axis, and rather is commonly defined by multiple parallel axes. Thus, x-axis is a specific axis defined in space, while X direction refers to the direction of the x-axis or any other axis that is parallel to the x-axis. Different but parallel axes can have the same X direction. Direction only has an orientation and not a location in space. In at least some embodiments, and with particular reference to FIG. 9, a coordinate system is presented with the x-axis coinciding with an axis of a shaft of the handheld electrosurgical tool, the y-axis oriented relative thereto, and the z-axis coming out of the paper.

Serial Kinematic Joint/Mechanism—The term “kinematics” may refer to the geometric study and description of motion of bodies relative to other bodies. A serial kinematic (SK) joint, or serial kinematic mechanism, consists of bodies connected via a serial chain of connectors, joints, or mechanisms. If one traces or scribbles a line from one body to another in a serial kinematic joint/mechanism, there exists only one mechanical path (or line) of motion transmission. In a somewhat simplistic example of a serial kinematic joint/mechanism, a first body and a second body are connected to each other via four connectors and three intermediate bodies. The first body and second body may be considered rigid, and the intermediate bodies may be considered rigid for practical purposes. The connectors may be simple or complex joints that may allow certain motions while constraining other motions. The connectors and intermediate bodies may span in-what-is-effectively a single line and mechanical path between the first and second bodies.

Parallel Kinematic Joint/Mechanism—In a somewhat simplistic example of a parallel kinematic (PK) mechanism, a first body is connected to a second body via multiple independent chains and lines of intermediate bodies. Each such chain represents a mechanical path of motion transmission. If one traces possible lines from the first body to the second body, there is more than one mechanical path, which makes this a parallel design. The connection paths are not parallel in a geometric sense (i.e., two straight lines being parallel such as the opposing sides of a rectangle), but parallel in the kinematic sense, which implies multiple (more than one), independent, non-overlapping chains or paths between the first body and second body. The connectors here are simple or complex joints that may allow certain motions and constrain other motions. For convenience, the term joint and connector may be used interchangeably.

End Effector Assembly—When provided in an embodiment, the end effector (EE) assembly may be referred to as the EE assembly. In some embodiments, the EE assembly may exist at the distal end of the tool shaft. An EE assembly may be one of a variety of types that are useful in a surgical arena (e.g., needle holder, hook electrode, grasper, dissector, forceps, vessel sealer, clip applier, etc.). The EE assembly may be coupled to the device frame (e.g., shaft) such that it is a rigid extension of the device. Alternatively, the EE assembly may be coupled to the device frame (e.g., shaft) via an output articulation joint.

Device Frame—When provided in an embodiment, the device frame refers to a structural body or subassembly, which may have relative motion amongst components, that may be part of a tool apparatus or surgical tool. In certain tool apparatuses, it may be connected to a handle assembly and/or an elongated device shaft. Terms, namely “device frame” and “frame,” may be used interchangeably throughout the document.

Device Shaft—When provided in an embodiment, a device shaft is generally a rigid extension of the frame, at its proximal end, which is a slender and elongated member, commonly a cylinder. An output body, such as an end-effector assembly, may be constrained to the distal end of the device shaft. The tool shaft may simply be referred to as the shaft. The axis of the tool shaft may be referred to as Device Shaft Axis throughout the description.

Energy Connector/Power Connector—When provided in an embodiment, the terms “energy connector” and “power connector” and “electrical connector” refer to a standard male or female type connector designed for maintaining a constant electrical connection between the connector and an adjacent electrically conductive body. The energy input to the device and this connector may be monopolar or bipolar energy. In some embodiments, an energy connector could be a male or female type banana plug connector.

Energy Path—In several embodiments, electrical energy will be input (i.e., supplied) to the embodiment through an energy connector. An energy path comprises the conductive bodies and electrical joints that allow for the transmission of continuous electrical energy.

Unlimited Roll—An apparatus (i.e., instrument, device, tool, system, mechanism, etc.) that provides unrestricted roll, or rotation about a single axis, of a body within the apparatus. Bodies that have unlimited roll do not have a limitation in the amount of roll allowed. The roll may also be referred to as “infinite” or unrestrained, or full rotational capabilities.

User Interface—A user interface serves as an input interface that a user interacts with to produce certain output at a remotely-located end of a machine or instrument or mechanism. User interface is generally an ergonomic feature on a body, which is part of an instrument, that is triggered by the user.

Channels of Data—Refers to the transfer of various types of data that may be communicated (input or output) through a connection. Examples include monopolar electrosurgical RF energy, bipolar electrosurgical RF generator, control of an electrosurgical generator (activate/deactivate), output body sensor data such as end effector jaw force, position, velocity, impedance, haptic feedback, or tissue sensing, control of distal articulation joints, controls for end effector mechanical joints, control of distally located motors or switches, control or distally located bodies, or control of remote bodies.

With reference now to the figures, embodiments of an electrical energy connection assembly 10 are described and depicted herein. The electrical energy connection assembly 10 can be equipped in a handheld electrosurgical tool 12 for cauterization of human tissue according to an example application of a medical device, among other potential applications some of which are presented below. In the handheld electrosurgical tool 12—and unlike past instruments with a fully constrained and/or rigidly connected electrical cord—the electrical energy connection assembly 10 furnishes a rotational degree of freedom between an accompanying electrical connector and components of the handheld electrosurgical tool 12 at or near a site of electrical energy conduction and delivery. Moreover, the electrical energy conduction and delivery facilitated via the electrical energy connection assembly 10 is substantially or wholly uninterrupted and maintained (i.e., when intended) during rotational movements about the rotational degree of freedom and that occur at the electrical connection assembly 10. Drawbacks encountered with the past instruments such as torque buildup, inhibited instrument maneuverability, visual or physical obstruction, and/or unwanted disconnections, are altogether avoided with use of the electrical energy connection assembly 10. Ergonomic and usability benefits are gained by facilitating cable connection at any rotational degree about the rotational degree of freedom. A more effective and efficient electrical energy connection is hence provided. Furthermore, the electrical energy connection assembly 10 can have various designs, constructions, and components in various embodiments depending upon-among other possible factors—the application in which the electrical energy connection assembly 10 is employed in use and the desired attributes of electrical energy transference.

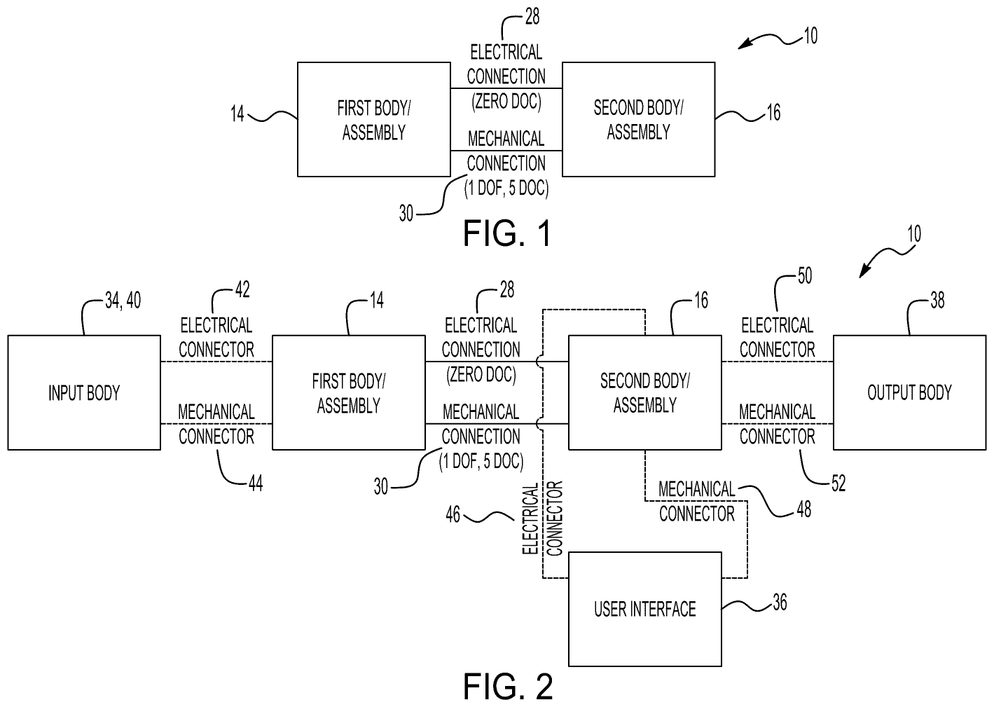

FIG. 1 presents an embodiment of the electrical energy connection assembly 10 in block diagrammatic form. Here, the electrical energy connection assembly 10 has a first body or assembly 14 and a second body or assembly 16. Depending on the embodiment and application of the electrical energy connection assembly 10, the first body/assembly 14 can be a single, discrete body or an assembly of multiple discrete bodies joined together in various ways. Likewise, the second body/assembly 16 can be a single, discrete body or an assembly of multiple discrete bodies joined together in various ways. In the example application of the handheld electrosurgical tool 12, for instance, and as depicted and described elsewhere, the first body/assembly 14 can be a rotatable energy hub assembly 18. Still, the first body/assembly 14 can be a first electrically conductive body 20 in the handheld electrosurgical tool application or in another application. The second body/assembly 16 can be a component of a user input assembly 22 such as a frame assembly 24 in the handheld electrosurgical tool 12, and/or can be an electrically nonconductive body 26 in the handheld electrosurgical tool 12 or in another application. In general, the phrase “electrically conductive,” and grammatical variations thereof, as used herein is intended to indicate an object or type of material that allows the flow of electric current in one or more directions. Examples of electrical conductive materials include copper and aluminum, among other metals, as well as nonmetallic materials like conductive polymers. Conversely, the phrase “electrically nonconductive,” and grammatical variations thereof, as used herein is intended to indicate an object or type of material that prevents the flow of electric current therethrough. Examples of electrical nonconductive materials include nonconductive plastic materials.

With continued reference to FIG. 1, in this embodiment an electrical connection 28 and a mechanical connection 30 resides between the first body/assembly 14 and the second body/assembly 16. The electrical connection 28 can take different forms in different embodiments. Here, the electrical connection 28 provides an electrical energy transference between the first body/assembly 14 and the second body/assembly 16. The electrical energy transference can be a monopolar electrical energy transference per one embodiment, or can be a bipolar electrical energy transference per another embodiment. The direction of electrical energy transference can be unidirectional and from the first body/assembly 14 to the second body/assembly 16, or can be bidirectional and from/to the first body/assembly 14 to/from the second body/assembly 16. No degrees of constraint are provided between the first body/assembly 14 and the second body/assembly 16 via the electrical connection 28, per this embodiment.

Further, the mechanical connection 30 can take different forms in different embodiments. In the embodiment of FIG. 1, the mechanical connection 30 furnishes a single degree of freedom between the first body/assembly 14 and the second body/assembly 16, and furnishes five degrees of constraint between the first body/assembly 14 and the second body/assembly 16. This motion and these constraints can be provided in various ways. In an embodiment, the single degree of freedom is a rotational degree of freedom of the first and second bodies/assemblies 14, 16 relative to each other, while the five degrees of constraint serve to constrain the remaining motions of the first and second bodies/assemblies 14, 16 relative to each other including translational motions therebetween. The rotational degree of freedom can be about an axis of the second body/assembly 16, such as a shaft axis (introduced below) or an axis of the user input assembly 22 in the example application of the handheld electrosurgical tool 12. In an embodiment, the rotational degree of freedom can provide full rotational capabilities between the first and second bodies/assemblies 14, 16 with respect to each other in which unlimited and unrestricted roll and rotation about the associated axis is provided. Conversely, in another embodiment, the rotational degree of freedom can provide less than full rotational capabilities between the first and second bodies/assemblies 14, 16 with respect to each other in which limited and restricted roll and rotation to a desired degree about the associated axis is provided. The full rotational capabilities can be provided in both the clockwise and counterclockwise rotational directions or in only one of the directions; or, the limited rotational capabilities can be provided in both the clockwise and counterclockwise rotational directions or in only one of the directions. Still further, in an embodiment, the rotational degree of freedom can be established via the first electrically conductive body 20 and the electrically nonconductive body 26; in another embodiment, the rotational degree of freedom can be established via an electrically nonconductive body 32 of the first body/assembly 14.

Furthermore, in an embodiment, the five degrees of constraint between the first body/assembly 14 and the second body/assembly 16 include three translational degrees of constraint and two rotational degrees of constraint, and can be provided between various bodies and/or assemblies of the first and second bodies/assemblies 14, 16. In embodiments, the five degrees of constraint can be effected via electrically conductive bodies and/or electrically nonconductive bodies of the first and second bodies/assemblies 14, 16. In an embodiment, as an example, the five degrees of constraint can be established via the first electrically conductive body 20 and the electrically nonconductive body 26; in another embodiment, the five degrees of constraint can be established via the electrically nonconductive body 32 of the first body/assembly 14. Still further, in an embodiment, the first body/assembly 14 can have a slip fit with respect to the second body/assembly 16.

FIG. 2 presents an embodiment of the electrical energy connection assembly 10 in block diagrammatic form. Here, in addition to the first and second bodies/assemblies 14, 16, the electrical energy connection assembly 10 has an input body 34, a user interface 36, and an output body 38.

The input body 34 may also be referred to as an energy input body or an energy generator in certain embodiments. The term “input” as used here does not necessary imply a direction of electrical energy flow; that is, in certain embodiments electrical energy flows through the input body 34 in a first direction and electrical energy flows back through the input body 34 in a second, opposite direction. The input body 34 constitutes an external electrical energy source. The input body 34 serves to supply an input to the first body/assembly 14 amid use of the electrical energy connection 10. The input body 34 and the input itself can take different forms in different embodiments. In an embodiment, the input body 34 can be an electrosurgical generator 40 that provides monopolar electrical energy or bipolar electrical energy as the input. Further, in an embodiment, the input body 34 can supply a multitude of channels of data and/or commands as the input to the first body/assembly 14, and/or can receive a multitude of channels of data from downstream components of the electrical energy connection 10 such as from the output body 38; these examples are intended to be embraced by the phrase electrically conductive. For example, input from the input body 34 can serve to control the electrosurgical generator 40 and activate and deactivate the monopolar or bipolar electrical energy provided thereby. In examples, the multitude of channels of data and/or commands can signal an end effector jaw force, position, velocity, impedance, and/or tissue sensing, in the handheld electrosurgical tool application or in another application. In another example, input from the input body 34 can serve to control electric motors situated at downstream components of the electrical energy connection 10 such as at the second body/assembly 16 and at the output body 38 to impart certain regulated movements thereat and thereof; in the handheld electrosurgical tool application or in another application, the imparted movements can be of an associated shaft and/or end effector. In a further example, haptic feedback can be received from components downstream of the input body 34 in the electrical energy connection 10 such as from the second body/assembly 16 and/or from the output body 38; in the handheld electrosurgical tool application or in another application, the haptic feedback can be prompted via certain movements and manipulations of an associated end effector assembly (introduced below). Yet further, in an embodiment, a foot pedal with a switch can be electrically coupled to the input body 34; here, the foot pedal could serve to activate and deactivate the monopolar or bipolar electrical energy provided by the input body 34 or could otherwise serve to manage the input from the input body 34. Lastly, according to an embodiment, the input body 34 has a connector for making a connection with the first body/assembly 14. In FIG. 2, the connector is in the form of an electrical connector 42 and a mechanical connector 44. The electrical connector 42 and mechanical connector 44 can be provided by component and body in at least some embodiments; for example, a male or female banana plug provides both a mechanical connection and an electrical connection and hence can constitute both of the electrical and mechanical connectors 42, 44 in this regard. In varying embodiments, the connector can provide the monopolar or bipolar electrical energy and/or can supply a multitude of channels of data and/or commands and/or can receive a multitude of channels of data. The connector can be a male or female banana plug, a multi-channel data input connector, and/or a power cable connector.

Still referring to FIG. 2, the user interface 36 serves as a way for a user to interface with the electrical energy connection assembly 10 for certain outputs and manipulations at a remotely-located end of a machine or instrument or mechanism. The user interface 36 can take different forms in different embodiments. In the embodiment of the figures, the user interface 36 has a connection with the second body/assembly 16. Here, the connection is in the form of an electrical connector 46 and a mechanical connector 48. The connection can constrain the user interface 36 and second body/assembly 16 together whereby roll and rotational motion is transmitted from the user interface 36 and to the second body/assembly 16. In an embodiment, the user interface 36 can have a switch situated thereon and coupled thereto. Here, the user of the electrical energy connection assembly 10 in the larger application can manage the input body 34 via the switch such as activating and deactivating the monopolar or bipolar electrical energy provided thereby. The switch can generate a signal in this regard. The signal can be back-fed through the second body/assembly 16 via the electrical connector 46, through the first body/assembly 14 via the electrical connection 28, and to the input body 34 via the electrical connector 42; or, per an embodiment, the signal can be transmitted to the input body 34 by other ways such as via wireless transmission. In the handheld electrosurgical tool application or in another application, the user interface 36 can be a handle assembly (introduced below) that is gripped and manipulated by a surgeon or other user. In the handle assembly embodiment, the handle assembly can have a fixed portion or component and a rotatable portion or component. These portions or components, when provided, work to furnish certain motions of an associated frame portion or component. Further, in the handle assembly embodiment, the handle assembly can have a handle body and a dial (both introduced below). The handle body is held by a user, and the dial can be rotated with respect to the handle body by the user. The dial, per this embodiment, is fixed and constrained to an associated frame portion or component whereby rotation of the dial causes rotation of the frame portion or component. The rotation can be about the axis of the second body/assembly 16, such as the shaft axis. Moreover, in the handheld electrosurgical tool application or another application, and in the handle assembly embodiment, the user interface 36 can serve as a local ground for the second body/assembly 16 and allow for rigid body motions of the user interface 36 to be transmitted to the second body/assembly 16. For example, free space position and orientation of the handle body can have direct influence on the frame. Here, axial positions (X, Y, Z directions) of the handle body can impact the axial positions of the frame. And rotational motions of the handle body can impact rotation of the frame. But pitch and yaw orientations of the handle body do not impact the position of the frame, per this example embodiment.

In FIG. 2, the output body 38 serves as an end effector assembly (introduced below) of the electrical energy connection assembly 10 or of the larger application such as the handheld electrosurgical tool application or other application. Upstream bodies of the electrical energy connection assembly 10—e.g., second body/assembly 16, user interface 36, first body/assembly 14, and input body 34—can work together to effect an output at the output body 38. The output body 38 and the output itself can take different forms in different embodiments. The output can be rotational and translational movements, monopolar or bipolar electrical energy, or something else, per various examples. In the embodiment of the figures, the output body 38 has a connection with the second body/assembly 16. Here, the connection is in the form of an electrical connector 50 and a mechanical connector 52. In an embodiment, the output body 38 is situated at a distal end of the electrical energy connection assembly 10 relative to other bodies and components of the electrical energy connection assembly 10. In FIG. 2, for example, the output body 38 is located at a distal end of the second body/assembly 16. In a further embodiment, the output body 38 can be a remotely-located slave body or assembly. In the end effector assembly embodiment of the handheld electrosurgical tool application, the output body 38 is equipped to cauterize human tissue amid minimally invasive surgical (MIS) procedures and remote access surgical procedures. Here, the output body 38 can be a monopolar or bipolar electrical energy end effector assembly. In yet another embodiment, the output body 38 can have a probe electrode. Here, the probe electrode can be active or inactive, depending on a state of the electrosurgical generator 40, per this embodiment.

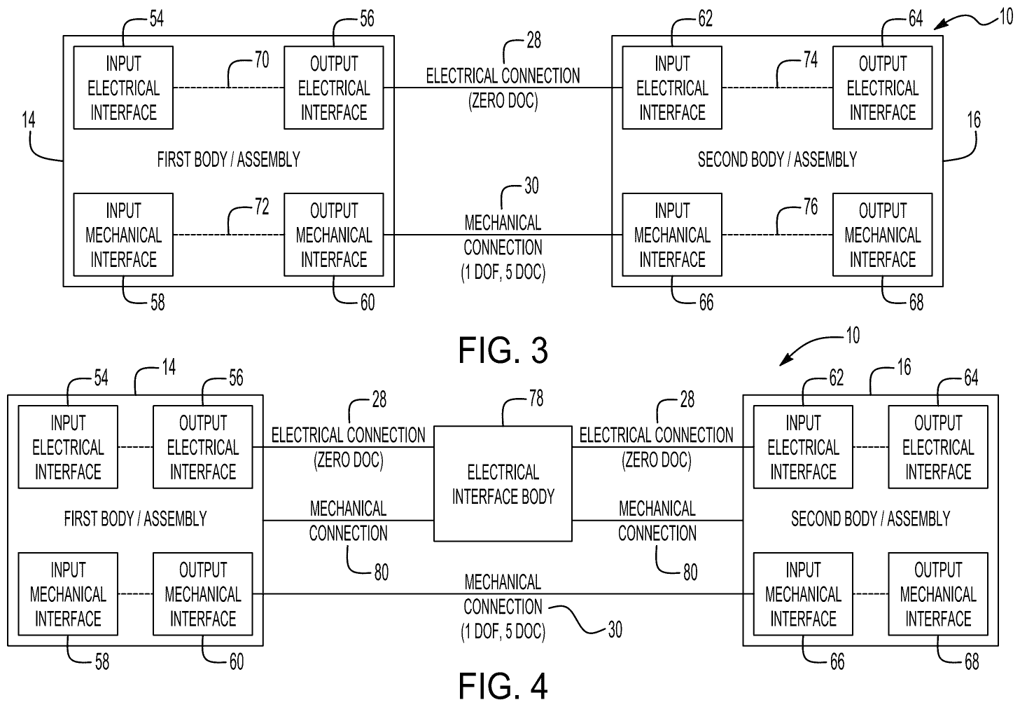

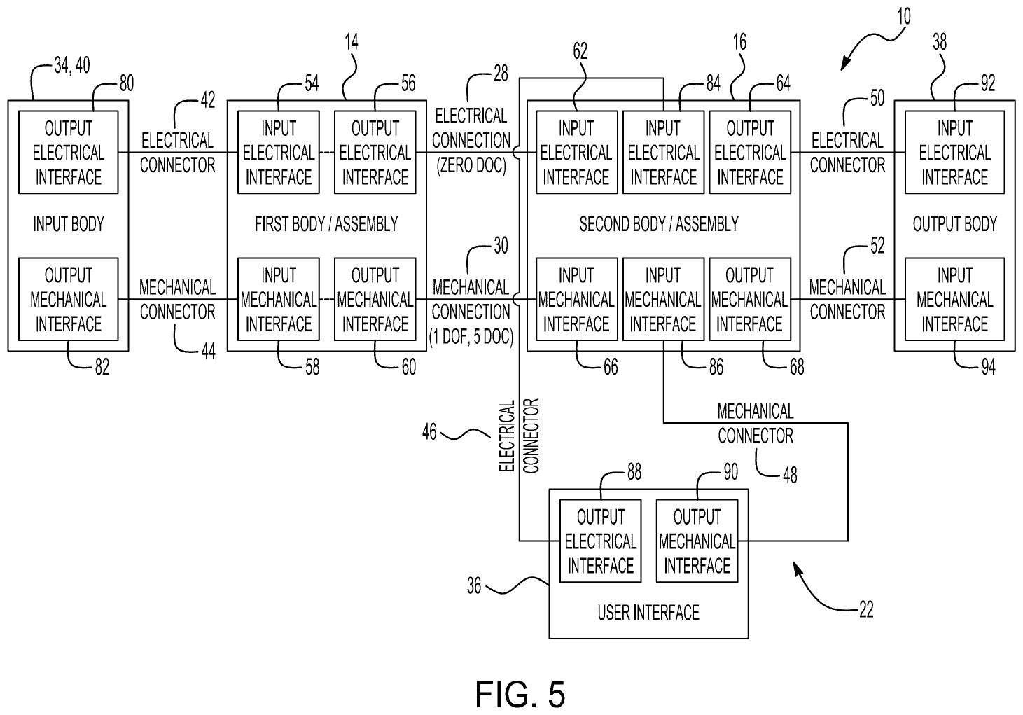

FIG. 3 presents an embodiment of the electrical energy connection assembly 10 in block diagrammatic form. Here, the first and second bodies/assemblies 14, 16 have input and output electrical and mechanical interfaces in order to exhibit electrical and mechanical interactions therebetween and with external bodies, and/or electrical and mechanical exchanges therebetween and with external bodies, among other potential interactions and/or exchanges and/or connections. In the embodiment of the figure, the first body/assembly 14 has a first input electrical interface 54, a first output electrical interface 56, a first input mechanical interface 58, and a first output mechanical interface 60. In a similar way, the second body/assembly 16 has a second input electrical interface 62, a second output electrical interface 64, a second input mechanical interface 66, and a second output mechanical interface 68. In this embodiment, the first input electrical interface 54 can interface with and have an electrical coupling with the input body 34 via the electrical connector 42, as depicted in FIG. 5. The first input electrical interface 54 can be a permanent connection with the first body/assembly 14, or can be a temporary connection of the first body/assembly 14 that can be selectively connected and disconnected. In varying embodiments, the first input electrical interface 54 can be constituted by a male or female banana plug, a multi-channel data input connector, a power cable connector, and/or some other type of electrical connector for the electrosurgical generator 40, as examples. Furthermore, in this embodiment the first output electrical interface 56 can interface with and have an electrical coupling with the second body/assembly 16 via the electrical connection 28. In varying embodiments, electrical energy can flow through the first output electrical interface 56, and/or one or more channels of data can flow through the first output electrical interface 56, and/or commands can flow through the first output electrical interface 56, among other possibilities. In the handheld electrosurgical tool application or in another application, the first output electrical interface 56 can be constituted by an inner housing or an energy hub (introduced below).

Moreover, in the embodiment of FIG. 3, a first conductive electrical energy path 70 resides between the first input electrical interface 54 and the first output electrical interface 56. The first conductive electrical energy path 70 can be referred to as a conductive path. The first conductive electrical energy path 70 can transmit electrical energy between the first input electrical interface 54 and the first output electrical interface 56, and/or can transmit one or more channels of data between the first input electrical interface 54 and the first output electrical interface 56, and/or can transmit commands between the first input electrical interface 54 and the first output electrical interface 56, among other possibilities. The first conductive electrical energy path 70 is initiated at the first input electrical interface 54 and terminates at the first output electrical interface 56, and spans therebetween. In varying embodiments, the first conductive electrical energy path 70 can be established via a point or line or surface contact and abutment interface between the first input electrical interface 54 and the first output electrical interface 56. Further, the first conductive electrical energy path 70 can be established within a single electrically conductive body; for example, the first conductive electrical energy path 70 can be constituted by a single electrically conductive wire or component; the first input and output electrical interfaces 54, 56 may then be on opposite ends of the electrically conductive body.

Further, in the embodiment of FIG. 3, the first input mechanical interface 58 can interface with and have a mechanical connection with the input body 34 via the mechanical connector 44, as depicted in FIG. 5. The first input mechanical interface 58 can provide some degree of retention for an output mechanical interface (introduced below) of the input body 34 via the mechanical connector 44; such retention can be permanent or temporary (e.g., snap fit, magnetic, etc.). In the handheld electrosurgical tool application or in another application, the first input mechanical interface 58 can be constituted by a banana plug power connector (introduced below). Furthermore, in this embodiment the first output mechanical interface 60 can interface with and have a mechanical connection with the second body/assembly 16 via the mechanical connection 30. In the handheld electrosurgical tool application or in another application, the first output mechanical interface 60 can be constituted by the energy hub or by an electrically nonconductive outer housing or cover (introduced below). A first mechanical transmission path 72 resides between the first input mechanical interface 58 and the first output mechanical interface 60 in the embodiment of FIG. 3.

Still referring to the embodiment of FIG. 3, the second input electrical interface 62 of the second body/assembly 16 can interface with and have an electrical coupling with the first body/assembly 14 and with the first output electrical interface 56 via the electrical connection 28. In the handheld electrosurgical tool application or in another application, the second input electrical interface 62 can be constituted by a conductor assembly (introduced below). Furthermore, in this embodiment the second output electrical interface 64 can interface with and have an electrical coupling with the output body 38 via the electrical connector 50. In the handheld electrosurgical tool application or in another application, the second output electrical interface 64 can be constituted by the conductor assembly. Moreover, in the embodiment of FIG. 3, a second conductive electrical energy path 74 resides between the second input electrical interface 62 and the second output electrical interface 64. The second conductive electrical energy path 74 can be referred to as a conductive path. The second conductive electrical energy path 70 can transmit electrical energy between the second input electrical interface 62 and the second output electrical interface 64, and/or can transmit one or more channels of data between the second input electrical interface 62 and the second output electrical interface 64, and/or can transmit commands between the second input electrical interface 62 and the second output electrical interface 64, among other possibilities. The second conductive electrical energy path 74 is initiated at the second input electrical interface 62 and terminates at the second output electrical interface 64, and spans therebetween. In varying embodiments, the second conductive electrical energy path 74 can be established via a point or line or surface contact and abutment interface between the second input electrical interface 62 and the second output electrical interface 64. Further, in varying embodiments, the second conductive electrical energy path 74 can be constituted by one or more bodies or carriers of electrical energy that are connected in a series arrangement and configuration, by an energy cable, by an electrically conductive wire such as a nitinol wire (introduced below), and/or by an electrically conductive shaft (introduced below).

Further, in the embodiment of FIG. 3, the second input mechanical interface 66 of the second body/assembly 16 can interface with and have a mechanical connection with the first body/assembly 14 via the mechanical connector 30. In the handheld electrosurgical tool application or in another application, the second input mechanical interface 66 can be constituted by a frame (introduced below). Furthermore, in this embodiment the second output mechanical interface 68 can interface with and have a mechanical connection with the output body 38 via the mechanical connection 52. In the handheld electrosurgical tool application or in another application, the second output mechanical interface 68 can be constituted by the shaft. A second mechanical transmission path 76 resides between the second input mechanical interface 66 and the second output mechanical interface 68 in the embodiment of FIG. 3.

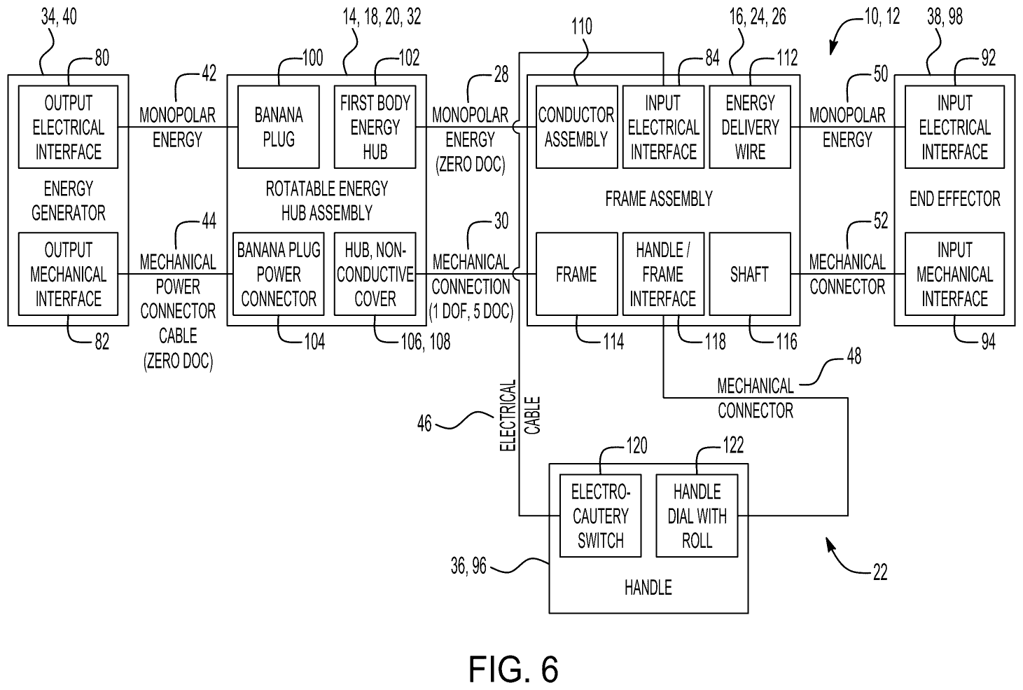

FIG. 4 presents an embodiment of the electrical energy connection assembly 10 in block diagrammatic form. Here, an electrical interface body 78 is provided and is situated between the first body/assembly 14 and the second body/assembly 16. The electrical interface body 78, when provided, serves as a connection between the first output electrical interface 56 of the first body/assembly 14 and the second input electrical interface 62 of the second body/assembly 16. The connection provided by the electrical interface body 78 can be electrical by way of the electrical connection 28, and can be mechanical by way of a mechanical connection 80 in the figure. In various embodiments, the electrical interface body 78 can transmit electrical energy between the first output electrical interface 56 and the second input electrical interface 62, and/or can transmit one or more channels of data between the first output electrical interface 56 and the second input electrical interface 62, and/or can transmit commands between the first output electrical interface 56 and the second input electrical interface 62, among other possibilities. Still, in various embodiments, the electrical interface body 78 can be a subcomponent or sub-body of the first body/assembly 14 or of the second body/assembly 16; and/or the electrical interface body 78 can be captured by, but not necessarily constrained to, the first body/assembly 14 or the second body/assembly 16. Further, with reference now to FIG. 6, in the handheld electrosurgical tool application or in another application, the electrical interface body 78 can be situated between the rotatable energy hub assembly 18 and the frame assembly 24. Further, in these exemplary applications, the electrical interface body 78 can be situated between the energy hub of the first body/assembly 14 and the conductor assembly of the second body/assembly 16.

The electrical connection furnished by the electrical interface body 78, per at least some embodiments, is a continuous electrical connection maintained between the first body/assembly 14 and the second body/assembly 16. In these embodiments, the continuous electrical connection remains thoroughly intact and uninterrupted and consistent while relative movement occurs between the first body/assembly 14 and the second body/assembly 16 (i.e., when intended). In a particular scenario, the continuous electrical connection remains uninterrupted amid rotational movements about the rotational degree of freedom exhibited between the first and second bodies/assemblies 14, 16; moreover, the continuous electrical connection remains uninterrupted amid unlimited rotations about the rotational degree of freedom. By way of this maintained electrical connection, a constant and consistent electrical energy conduction and transference remains in effect between the first and second bodies/assemblies 14, 16 despite relative movements therebetween. In varying embodiments and applications-including in the handheld electrosurgical tool application or in another application—there can be one or more mechanical points or lines or surfaces maintained between the first and second bodies/assemblies 14, 16; the maintained electrical connection should remain during use of the handheld electrosurgical tool and during reprocessing of the handheld electrosurgical tool; and/or the maintained electrical connection should remain throughout the useful life of the handheld electrosurgical tool.

The maintained electrical connection can be provided in varying ways according to various embodiments. In an embodiment, the electrical interface body 78 exerts a force to the first body/assembly 14, to the second body/assembly 16, or to both the first and second body/assembly 14, 16. The exertion of force can be at a single location or at a multitude of locations. The electrical interface body 78 can be a preloaded spring. Further, the electrical interface body 78 can be a biasing member that is situated at or near the first output electrical interface 56 and the second input electrical interface 62; the biasing member can exert a force to, and maintain contact with, the first output electrical interface 56 and second input electrical interface 62. The electrical interface body 78 can be a canted coil spring or a garter spring, per various embodiments. Here, the canted coil spring or garter spring can be constrained between the first body/assembly 14 and the second body/assembly 16. The canted coil spring or garter spring can concurrently maintain numerous points of contact and load between both of the first and second bodies/assemblies 14, 16, and thereby can provide a constant and consistent electrical energy transference path therebetween. In this embodiment, the electrical energy flow is directed generally radially between the first body/assembly 14 and the second body/assembly 16 via the canted coil spring or garter spring. Furthermore, per various embodiments, the electrical interface body 78 can be a wave spring. The wave spring can be situated between the first body/assembly 14 and the second body/assembly 16, and can exert a force and load between the first body/assembly 14 and second body/assembly 16 and thereby maintain contact therebetween. The exerted force and load in the embodiment of the wave spring can be in an axial direction. As before, by way of the maintained contact, the wave spring provides a constant and consistent electrical energy transference path between the first body/assembly 14 and the second body/assembly 16. Still further, per various embodiments, the electrical interface body 78 can be an electrically conductive bearing or bushing. Yet further, per various embodiments, the electrical interface body 78 can be a pogo pin or spring-loaded pin. The pogo pin can be situated between the first body/assembly 14 and the second body/assembly 16, and can exert a force and load between the first body/assembly 14 and second body/assembly 16 and thereby maintain contact therebetween. The exerted force and load in the embodiment of the pogo pin can be in a radial direction. As before, by way of the maintained contact, the pogo pin provides a constant and consistent electrical energy transference path between the first body/assembly 14 and the second body/assembly 16.

FIG. 5 presents an embodiment of the electrical energy connection assembly 10 in block diagrammatic form. Here, the input body 34 has a third output electrical interface 80 and a third output mechanical interface 82; the second body/assembly 16 has a third input electrical interface 84 and a third input mechanical interface 86; the user interface 36 has a fourth output electrical interface 88 and a fourth output mechanical interface 90; and the output body 38 has a fourth input electrical interface 92 and a fourth input mechanical interface 94. The third output electrical interface 80 of the input body 34 can interface with and have an electrical coupling with the first body/assembly 14 and with the first input electrical interface 54 via the electrical connector 42. Similarly, the third output mechanical interface 82 of the input body 34 can interface with and have a mechanical connection with the first body/assembly 14 and with the first input mechanical interface 58 via the mechanical connector 44. Further, the third input electrical interface 84 of the second body/assembly 16 can interface with and have an electrical coupling with the user interface 36 and with the fourth output electrical interface 88 via the electrical connector 46. Similarly, the third input mechanical interface 86 of the second body/assembly 16 can interface with and have a mechanical connection with the user interface 36 and with the fourth output mechanical interface 90 via the mechanical connector 48. Yet further, the fourth input electrical interface 92 of the output body 38 can interface with and have an electrical coupling with the second body/assembly 16 and with the second output electrical interface 64 via the electrical connector 50. And similarly, the fourth input mechanical interface 94 of the output body 38 can interface with and have a mechanical connection with the second body/assembly 16 and with the second output mechanical interface 68 via the mechanical connector 52.

FIG. 6 presents an embodiment of the electrical energy connection assembly 10 and of the handheld electrosurgical tool 12 in block diagrammatic form. This embodiment presents example components in the handheld electrosurgical tool application or in another application; still, more, less, and/or different components can be provided in various embodiments. In the embodiment of FIG. 6, the input body 34 is in the form of the electrosurgical generator 40, the first body/assembly 14 is in the form of the rotatable energy hub assembly 18, the second body/assembly 16 is in the form of the frame assembly 26, the user interface 36 is in the form of a handle 96, and the output body 38 is in the form of an end effector 98. The end effector 98 can be an electrically conductive end effector that is composed of an electrically conductive material such as a metal material. In this embodiment, the electrical connector 42 furnishes monopolar electrical energy, and the mechanical connector 44 is in the form of a mechanical power connector cable. Further, the first input electrical interface 54 of the rotatable energy hub assembly 18 is in the form of a banana plug 100, and the first output electrical interface 56 is in the form of a first body energy hub 102. The first input mechanical interface 58 is in the form of a banana plug power connector 104, and the first output mechanical interface 60 is in the form of an energy hub 106 and/or an electrically nonconductive cover 108. Between the rotatable energy hub assembly 18 and the frame assembly 26, the electrical connection 28 furnishes monopolar electrical energy, per this embodiment. Furthermore, at the frame assembly 26, the second input electrical interface 62 is in the form of a conductor assembly 110; the second output electrical interface 64 is in the form of an energy delivery wire 112; the second input mechanical interface 66 is in the form of a frame 114; the second output mechanical interface 68 is in the form of a shaft 116; and the third input mechanical interface 86 is in the form of a handle/frame interface 118. Between the frame assembly 26 and the handle 96, the electrical connector 46 is in the form of an electrical cable, per this embodiment. Further, at the handle 96, the fourth output electrical interface 88 is in the form of an electrocautery switch 120, and the fourth output mechanical interface 90 is in the form of a handle dial 122. The handle dial 122 can exhibit roll capabilities per an embodiment, though need not exhibit roll capabilities in other embodiments. Lastly, in the embodiment of FIG. 6, between the frame assembly 26 and the end effector 98, the electrical connector 50 furnishes monopolar electrical energy.

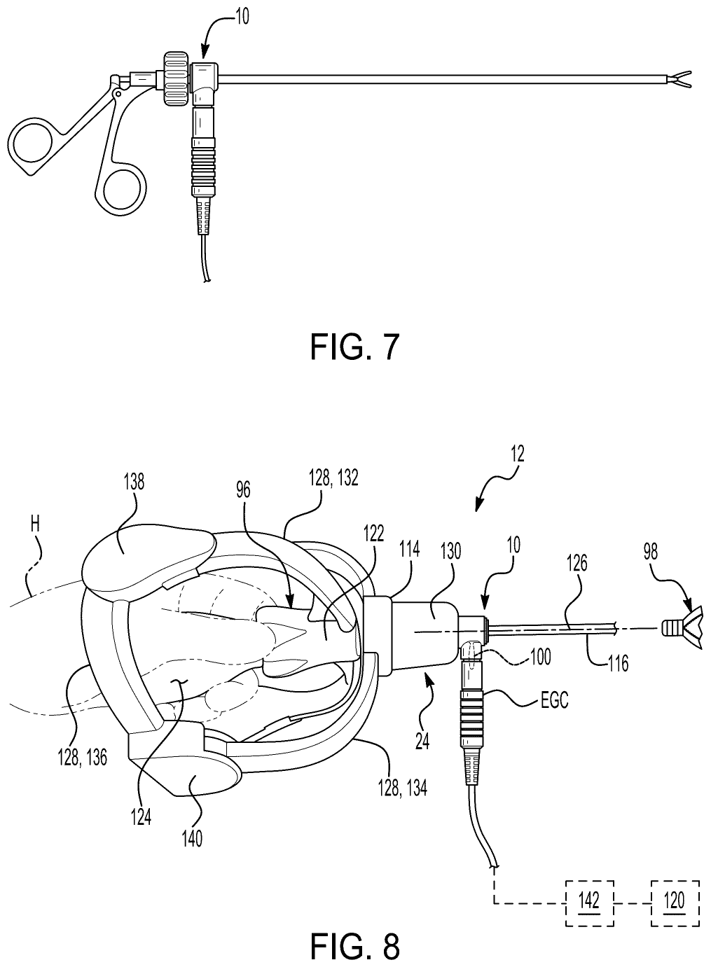

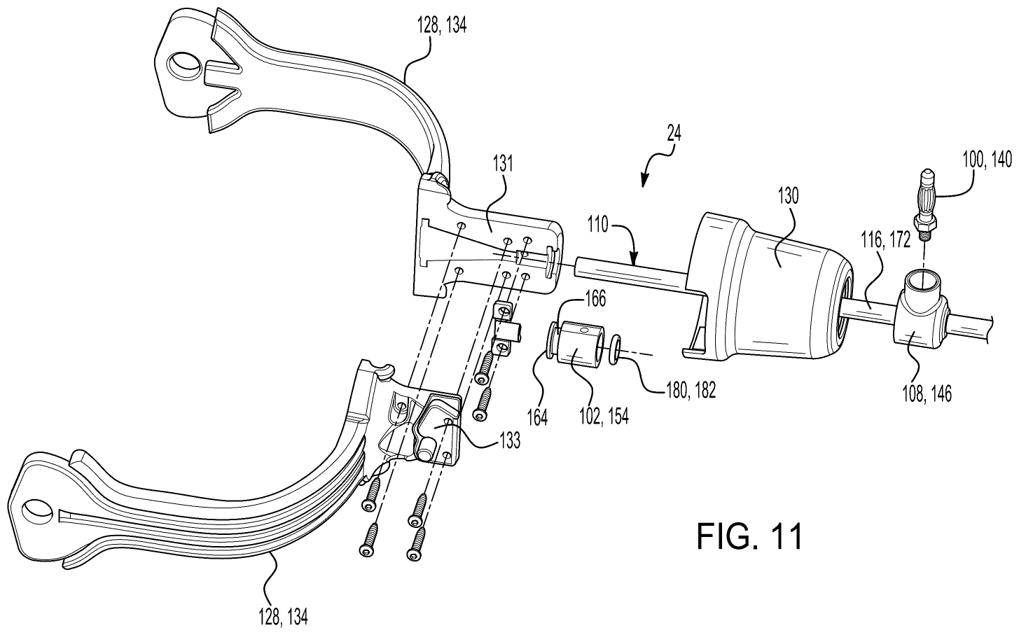

FIGS. 8-20 present various embodiments of the electrical energy connection assembly 10 implemented in the handheld electrosurgical tool application and in a medical device application. The handheld electrosurgical tool 12 can be employed for use in minimally invasive surgical (MIS) procedures and remote access surgical procedures, among other potential procedures. Still, while presented in the context of the handheld electrosurgical tool 12, the designs, constructions, and components described in connection with FIGS. 8-20 are applicable and can be implemented in other applications including other medical and non-medical applications. In the example of FIG. 7, the electrical energy connection assembly 10 is equipped for use in a laparoscopic instrument with a scissor-like handle and end effector. In general, the electrical energy connection assembly 10 can be implemented and equipped in devices that lack articulation capabilities at an end effector and at an associated handle. As an example of a non-medical application, the electrical energy connection assembly 10 can be implemented and equipped in an industrial application such as a biosafety and/or radioactive cabinet device for manipulation of items therein via such a device. Yet still, other applications are possible.

With particular reference to FIG. 8, an example of the handheld electrosurgical tool 12 is partially depicted. In general, a user such as a surgeon grabs and holds the handle 96 of the handheld electrosurgical tool 12 with their hand H (illustrated in broken lines) and manipulates the handle 96 in order to effect intended movements and actions at the end effector 98 of the handheld electrosurgical tool 12. Movements and actions inputted at the handle 96 are transmitted to the end effector 98. The handheld electrosurgical tool 12 further has the frame assembly 24, frame 114, and the shaft 116. In at least certain embodiments, the end effector 98 extends from a distal end of the shaft 116. Components, parts, and portions of the handle 96 and/or of the frame assembly 24 can constitute the user input assembly 22 in the embodiments of FIGS. 8-20. Example handle assemblies (i.e., handle), frame assemblies, and end-effector assemblies (i.e., end effector) are depicted and described in U.S. Pat. No. 11,950,966 issued on Apr. 9, 2024 and owned by present applicant FlexDex, Inc., the contents of which are hereby incorporated by reference in their entirety. Further examples of handle assemblies, frame assemblies, and end-effector assemblies are depicted and described in U.S. Patent Application Publication No. 2023/0040475 published on Feb. 9, 2023 and owned by present applicant FlexDex, Inc., the contents of which are hereby incorporated by reference in their entirety. An energy generator connector cable EGC establishes a connection with the electrical energy connection assembly 10 amid use of the handheld electrosurgical tool 12. The supply and activation of external electrical energy to the electrical energy connection assembly 10 is via the electrocautery switch 120 which can be a foot pedal that is accessible and actuatable by the user of the handheld electrosurgical tool 12. The foot pedal can be actuated by the user's foot. In the foot pedal example, the foot pedal controls the flow of electrical energy prior to travel via the energy generator connector cable EGC. In another example, the electrocautery switch 120 can be device-mounted and accessible by the user's hand H. Here, the electrocautery switch 120 can be mounted on the user input assembly 22 such as at the handle 96.

In the embodiment of FIG. 8, the handle 96 includes the handle dial 122 and a handle body 124. The handle dial 122 serves as a user input for effecting unlimited roll functionality and capabilities. The roll functionality is with respect to and about a shaft axis 126, and is transmitted to the end effector 98 via intermediate bodies 128, and the shaft 116. A shaft mount housing 130 and shaft mount structures 131, 133 are situated at a proximal end of the shaft 116 and can be part of the frame assembly 24. The handle dial 122 rotates with respect to the handle body 124. The intermediate bodies 128 are rigid in construction and three in quantity: a first intermediate body or first half ring 132, a second intermediate body or second half ring 134, and a third intermediate body or deviation ring or full ring 136. The first half ring 132 extends from the handle dial 122 and has a fixed connection therewith. Similarly, the second half ring 134 extends from the shaft mount structures 131, 133 and has a fixed connection therewith. The full ring 136, on the other hand, is joined to the first half ring 132 via a first joint which can be a set of pin joints, and is joined to the second half ring 134 via a second joint which can be a set of pin joints. The first joint provides a yaw degree of freedom between the full ring 136 and the first half ring 132, and the second joint provides a pitch degree of freedom between the full ring 136 and the second half ring 134. Furthermore, according to this embodiment, a first pulley 138 is situated at one of the pin joints of the first joint and serves to capture yaw rotation of the first half ring 132 with respect to the full ring 136, and a second pulley 140 is situated at one of the pin joints of the second joints and serves to capture pitch rotation of the second half ring 134 with respect to the full ring 136. The captured yaw rotation is transmitted to the end effector 98 via control cables, and likewise the captured pitch rotation is transmitted to the end effector 98 via another set of control cables; still, transmission of motion can be carried out in other ways in other embodiments such as via digital signals among sensors and end effector drive motors, as an example. Components of the handle 96 and frame assembly 24—e.g., the handle dial 122, handle body 124, intermediate bodies 128, shaft mount housing 130, and shaft mount structures 131, 133—13 are typically composed of an electrically nonconductive material. Nonconductive materials are typically present on exterior portions of the handheld electrosurgical tool 12 that could come into contact with the user, a patient, or another medical device or equipment being used concurrently in operation to prevent unwanted electrical energy transmission therewith.

With particular reference now to FIGS. 9-12, a first embodiment of the electrical energy connection assembly 10 includes the rotatable energy hub assembly 18 (also called an infinitely rotatable energy hub assembly) and includes the frame assembly 24; still, more, less, and/or different components could be provided in alternative designs and constructions of the electrical energy connection assembly 10, at least some of which are depicted and described in subsequent embodiments. The rotatable energy hub assembly 18 and the frame assembly 24 can have varying designs, constructions, and components in various embodiments. In the first embodiment, the rotatable energy hub assembly 18 serves to facilitate a continuous electrical connection that remains substantially uninterrupted amid roll and rotational movements of the frame assembly 24 and of the shaft 116 and of the accompanying end effector about the shaft axis 126; still, in certain embodiments the electrical connection could be intentionally intermittent in nature. Relative to the larger handheld electrosurgical tool 12, the rotatable energy hub assembly 18 is located adjacent the frame assembly 24 and adjacent the proximal end and mounting region of the shaft 116. In this embodiment, the rotatable energy hub assembly 18 includes the banana plug 100, the electrically nonconductive cover 108 (or electrically non-conductive outer housing or insulative energy hub cover), and the first body energy hub 102 (or energy hub or rotatable energy hub or electrically conductive inner housing); still, the rotatable energy hub assembly 18 could have more, less, and/or different components in other embodiments.

The banana plug 100 serves as a power connector for the electrical energy connection assembly 10, according to this first embodiment. The banana plug 100 constitutes an electrical connector 140. The banana plug 100 receives connections with an external electrical energy source 142 such as via the energy generator connector cable EGC (e.g., see FIG. 8) for the provision of electrical energy to the electrical energy connection assembly 10. The banana plug 100 is carried by the electrically nonconductive cover 108 and by the first body energy hub 102, and is free to concurrently move and rotate therewith about the shaft axis 126. The connection with the energy generator connector cable EGC can also move and rotate therewith without an unintended disconnection. While able to rotate, the banana plug 100 can frequently remain directed vertically downward (i.e., see FIG. 8) due to the weight of the energy generator connector cable EGC or in an orientation dictated by routing of the energy generator connector cable EGC—this grants the user, such as the surgeon, the ability to use and manipulate the handle 96 without interference from the position of the energy generator connector cable EGC. A threaded mounting between the banana plug 100 and the first body energy hub 102 establishes a connection therebetween. Direct and immediate contact and connection between the banana plug 100 and the shaft 116 is absent at their confrontation shown in FIG. 10; rather, a slight clearance can reside therebetween. The banana plug 100 is itself electrically conductive and can be composed of an electrically conductive material such as a metal material.

The banana plug 100 in this embodiment is in the form of a male banana plug but could be a female banana plug; still, in yet other embodiments the electrical connector 140 could be a multi-channel data input connector, a power cable connector, and/or some other type of electrical connector. In the first embodiment, monopolar electrical energy provision and transference is furnished by the banana plug 100; still, in other embodiments bipolar electrical energy could be provided and/or a multitude of channels of data could be supplied and/or received via the electrical connector 140. Furthermore, according to this embodiment and with particular reference to FIG. 10, a longitudinal axis 144 is defined and established by a longitudinal extent of the banana plug 100. In this configuration of components of the electrical energy connection assembly 10, the longitudinal axis 144 is arranged unaligned with respect to an axis of rotation 145 of the rotatable energy hub assembly 18 which, according to this embodiment, is the shaft axis 126. More particularly, the longitudinal axis 144 has an orthogonal and perpendicular arrangement with respect to the axis of rotation 145. Still, in other embodiments, other non-orthogonal angles can be established between the longitudinal axis 144 and the axis of rotation 145 (e.g., the shaft axis 126). Lastly, the carrying and mounting of the banana plug 100 or other electrical connector 140 can be permanent or temporary and hence removable as desired.

With continued reference to FIGS. 9-11, the electrically nonconductive cover 108 serves to preclude and prevent unwanted and unintended electrical energy conduction and leakage to bodies at the exterior of the rotatable energy hub assembly 18. Electrical energy insulation is furnished via the electrically nonconductive cover 108. The electrically nonconductive cover 108 constitutes an electrically nonconductive housing 146. The electrically nonconductive cover 108 is shaped and sized to partially or more surround the banana plug 100 and to partially or more surround the first body energy hub 102. Here, an end region of the banana plug 100 remains exposed for accessibility outside of the electrically nonconductive cover 108, while the first body energy hub 102 is more largely surrounded by the electrically nonconductive cover 108 and otherwise lacks exposure exteriorly thereof. As illustrated best by FIG. 10, a first opening 148 accommodates passage of the banana plug 100, a second opening 150 accommodates passage of the shaft 116, and a rear open end 152 accommodates the first body energy hub 102. The electrically nonconductive cover 108 can be composed of an electrically nonconductive material such as a plastic material. Further, the electrically nonconductive cover 108 can fit closely over an exterior of the first body energy hub 102 and can be press-fit in place in installation, or could have a more positive interconnection with the first body energy hub 102 and/or with the shaft mount housing 130 and/or with the shaft mount structures 131, 133, as examples. In assembly, the electrically nonconductive cover 108 is constrained to the first body energy hub 102. Moreover, as described below, in the first embodiment the first body energy hub 102 furnishes a single rotational degree of freedom between the rotatable energy hub assembly 18 and the frame assembly 24 and furnishes five degrees of constraint therebetween, but in other embodiments the electrically nonconductive cover 108 could furnish the single rotational degree of freedom and the five degrees of constraint between the rotatable energy hub assembly 18 and the frame assembly 24. Still, in further embodiments the electrically nonconductive cover 108 could exhibit structures that furnish multiple degrees of constraint between it and the frame assembly 24.

In the first embodiment, the first body energy hub 102 serves to provide a path of electrical energy conduction and transference downstream of the banana plug 100 (the term downstream is used in this context, and more broadly herein, with reference to the flow of electrical energy from input to output; likewise, the term upstream is used herein with reference to same and is opposite in nature to downstream), and further serves to furnish the single rotational degree of freedom and the five degrees of constraint between the rotatable energy hub assembly 18 and the frame assembly 24. Electrical energy transference further occurs downstream of the first body energy hub 102 via the first body energy hub itself. The first body energy hub 102 constitutes an electrically conductive hub body 154. In assembly and installation, the first body energy hub 102 carries both the banana plug 100 and the electrically nonconductive cover 108. In the first embodiment, the first body energy hub 102 has an overall cylindrical shape. A central passage 156 spans axially through the cylindrical shape, as shown in FIG. 10, for accommodating receipt and insertion of the shaft 116. The first body energy hub 102 and the shaft 116 can exhibit a slip-fit relative to each other at the central passage 156. A threaded passage 158 accepts reception of the banana plug 100. Further, a recess 160 accepts seating of a spring (introduced below) in assembly and installation. In this embodiment, the first body energy hub 102 is composed of an electrically conductive material such as a metal material. Here, the first body energy hub 102 is made wholly of the electrically conductive material. Still, in other embodiments, the first body energy hub 102 could have certain portions composed of an electrically conductive material and other portions composed of an electrically nonconductive material; for example, one or more electrically conductive paths and one or more electrically nonconductive paths could be provided via the first body energy hub 102. In the first embodiment, monopolar electrical energy transference is furnished by the first body energy hub 102; still, in other embodiments bipolar electrical energy transference could be provided and/or a multitude of channels of data could be supplied and/or received by way of the first body energy hub 102.

As set forth, in this embodiment, a single rotational degree of freedom and five degrees of constraint between the rotatable energy hub assembly 18 and the frame assembly 24 are furnished via the first body energy hub 102. This can be effected by way of different designs, constructions, and components in different embodiments. With particular reference to FIG. 10, here, the first body energy hub 102 is constrained in part to the shaft mount structures 131, 133 and yet free to rotate relative thereto. A projection-recess interconnection 162 is established therebetween and constitutes the mechanical connection 30. The first body energy hub 102 has a first projection 164 and a first recess 166, and the shaft mount structures 131, 133 each have a corresponding second projection 168 and a second recess 170. The first projection 164 extends radially-outwardly and the second projections 168 extend radially-inwardly. The respective projections and recesses inter-engage with each other when the shaft mount structures 131, 133 are brought together in assembly. When engagement occurs, the first body energy hub 102 and shaft mount structures 131, 133 possess three translational degrees of constraint and two rotational degrees of constraint with respect to each other. The translational degrees of constraint include X, Y, and Z translational degrees of constraint; the X translational degree of constraint can correspond to constraint along the shaft axis 126. Still, the projection-recess interconnection 162 permits rotation R of the first body energy hub 102 with respect to the shaft mount structures 131, 133 about the shaft axis 126 in both the clockwise and counterclockwise directions and without limit. Here, the shaft axis 126 constitutes the axis of rotation 145 of the first body energy hub 102. Furthermore, the single rotational degree of freedom and five degrees of constraint between the rotatable energy hub assembly 18 and frame assembly 24 could be provided in other ways according to other embodiments. For example, a projection-recess interconnection could be established between the shaft mount housing 130 and the first body energy hub 102, between the electrically nonconductive cover 108 and the shaft mount structures 131, 133, or between the electrically nonconductive cover 108 and the shaft mount housing 130, among other possibilities. Yet further, the single rotational degree of freedom and five degrees of constraint could be provided by having the first body energy hub 102 in the form of two halves brought together around the shaft 116 with a lip-groove inter-engagement and inter-connection thereamong.

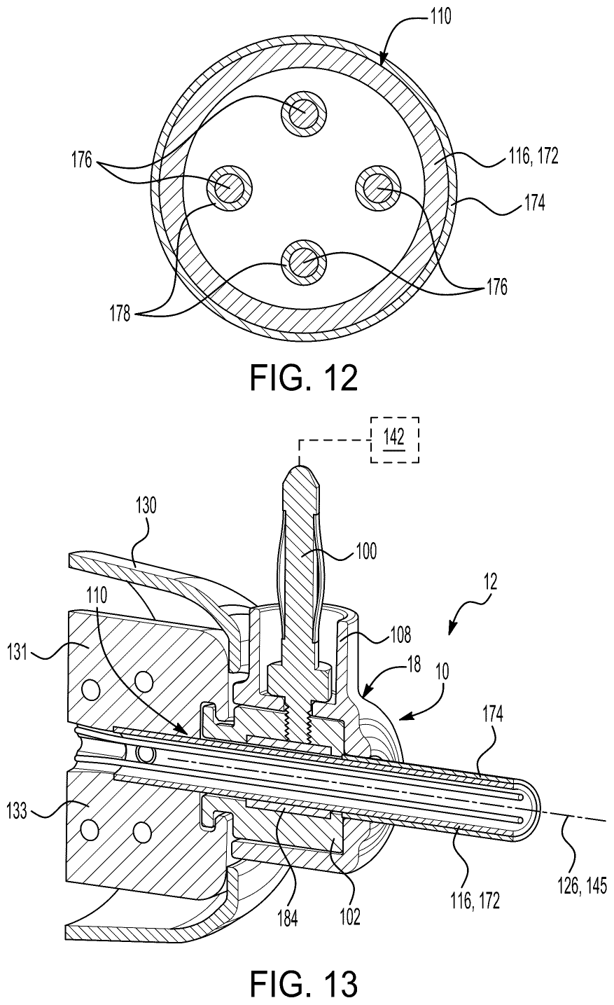

Still referring to the first embodiment of FIGS. 9-12, the frame assembly 24 here includes the conductor assembly 110. The conductor assembly 110 serves to provide a path of electrical energy transference downstream of the rotatable energy hub assembly 18 and to the accompanying end effector. The conductor assembly 110 can have varying designs, constructions, and components in various embodiments. In the first embodiment, the conductor assembly 110 includes an electrically conductive shaft 172 which is the shaft 116. The electrically conductive shaft 172 is composed of an electrically conductive material such as an electrically conductive thermoplastic or composite material, or a metal material like steel or stainless steel, or something else. In order to transfer electrical energy to the end effector, at a distal end of the electrically conductive shaft 172 adjacent the location of the end effector, an electrically conductive wire can be soldered or crimped or otherwise attached to the electrically conductive shaft 172 and connected to the end effector for downstream electrical energy transference thereto; still, alternative designs, constructions, and components could be employed for the transfer of electrical energy to the end effector which may be dictated by the end effector itself; for example, the end effector could itself be electrically conductive and could be in direct contact with the electrically conductive shaft 172, or an electrically conductive intermediate body could be situated between the electrically conductive end effector and the electrically conductive shaft 172. Furthermore, according to the first embodiment, an electrically nonconductive shaft sleeve 174 (or cover or sheath) is situated over an outer surface of the electrically conductive shaft 172 for the purpose of electrical energy insulation. The electrically nonconductive shaft sleeve 174 can be composed of an electrically nonconductive material such as a plastic material. The electrically nonconductive shaft sleeve 174 spans axially along the electrically conductive shaft 172 from the rotatable energy hub assembly 18 and to the end effector. Unwanted and unintended electrical energy conduction and leakage to bodies at the exterior of the electrically conductive shaft 172 is hence precluded and prevented via the electrically nonconductive shaft 174. Indeed, the electrically nonconductive shaft sleeve 174, as illustrated best by FIG. 10, spans into and within the electrically nonconductive cover 108 via the second opening 150 for increased prevention of electrical energy leakage. In different examples, the electrically nonconductive shaft sleeve 174 can take the form of an insulative heat shrink wrap, or an insulative coating applied to the electrically conductive shaft 172, or something else. The sectional view of FIG. 12 is perhaps the best illustration of the electrically nonconductive shaft sleeve 174 and its relation to the electrically conductive shaft 172. End effector control cables 176 are also shown in the figure. The end effector control cables 176 span from the handle 96 and to the accompanying end effector, and span and are routed through an axial extent of the shaft 116. Further here, electrically nonconductive cable sheaths 178 are situated over outer surfaces of the control cables 176 for the purpose of electrical energy insulation. The electrically nonconductive cable sheaths 178 can be composed of an electrically nonconductive material such as a plastic material.

Furthermore, in the first embodiment, a canted coil spring 180 (or garter spring) is provided and situated between the first body energy hub 102 and the electrically conductive shaft 172. The canted coil spring 180 serves to provide the continuous and maintained electrical connection between the first body energy hub 102 and electrically conductive shaft 172 for continuous and constant electrical energy conduction and transference therebetween (i.e., when intended), and ultimately to the accompanying end effector, that remains uninterrupted when the components rotate with respect to each other about the axis of rotation 145 (i.e., here, the shaft axis 126). The canted coil spring 180 is trapped in place at its location between the first body energy hub 102 and electrically conductive shaft 172, and establishes multiple points of contact with both components and exerts loads therebetween. It can rotate with the first body energy hub 102 and/or with the electrically conductive shaft 172 amid use of the handheld electrosurgical tool 12, or can remain static relative thereto. In the first embodiment, the canted coil spring 180 is the electrical interface body 78 and constitutes an electrically conductive biasing member 182. In assembly and installation, as perhaps demonstrated best by FIG. 10, the canted coil spring 180 is housed within the electrically nonconductive cover 108 and is disposed circumferentially around the electrically conductive shaft 172. The canted coil spring 180 is seated within the recess 160 of the first body energy hub 102 where it makes direct surface-to-surface contact with surfaces of the first body energy hub 102 and of the recess 160. Where the canted coil spring 180 is disposed over the electrically conductive shaft 172, the electrically conductive shaft 172 is free of the electrically nonconductive sleeve 174 whereby direct surface-to-surface contact is made between the shaft's electrically conductive outer surface and the canted coil spring 180. The canted coil spring 180 is radially sandwiched between the first body energy hub 102 and the electrically conductive shaft 172. Further, the canted coil spring 180 is itself electrically conductive and can be composed of an electrically conductive material such as a metal material. Yet further, due to its structure the canted coil spring 180 is made up of a multitude of loops, thereby furnishing a multitude of electrical energy conductive paths between the electrically conductive hub body 154 and the electrically conductive shaft 172; this has proven useful and beneficial per certain embodiments in the provision of the continuous and maintained electrical connection, and especially for facilitating and expanding longevity through reprocessing of the handheld surgical tool 12 and despite potential contaminant build-up.

In the first embodiment, electrical energy conduction and transference spans from the external electrical energy source 142 and to the accompanying end effector by way of the rotatable energy hub assembly 18, via the canted coil spring 180, and via the electrically conductive shaft 172. The flow of electrical energy transference can take the following path, per this embodiment: from the external electrical energy source 142, to and through the banana plug 100, to and through the first body energy hub 102, to and through the canted coil spring 180, to and through the electrically conductive shaft 172, and ultimately to the accompanying end effector; still, the flow of electrical energy transference could involve more, less, and/or different components including intervening components in variations to this first embodiment. The flow of electrical energy transference can involve monopolar electrical energy, bipolar electrical energy, and/or a multitude of channels of data. Moreover, the electrical energy conduction and transference remains intact and uninterrupted amid manipulations and movements of the handle 96 and of the frame assembly 24 that are imparted to the accompanying end effector including roll and rotational movements about the axis of rotation 145.

Turning now to FIG. 13, a second embodiment of the electrical energy connection assembly 10 is presented. In the depiction and description of the second embodiment, corresponding components and elements have similar reference numerals and indications as those in the first embodiment. Moreover, similarities may exist between the described first embodiment and the second embodiment, some of which might not be repeated here in the description of the second embodiment. At least certain appreciable differences between the embodiments are set forth.

In FIG. 13, the electrical interface body 78 is in the form of an electrically conductive bearing 184 (or bushing). The electrically conductive bearing 184 is situated between the first body energy hub 102 and the electrically conductive shaft 172. The electrically conductive bearing 184 has a sleeve-shaped construction that axially overlaps with the first body energy hub 102 and the electrically conductive shaft 172. The extent of axial overlap with the first body energy hub 102 can be a majority of a full axial extent of the first body energy hub 102, as demonstrated in FIG. 13 according to this second embodiment. The electrically conductive bearing 184-together with the first body energy hub 102-serves to provide the continuous and maintained electrical connection between the first body energy hub 102 and electrically conductive shaft 172 for continuous and constant electrical energy conduction and transference therebetween (i.e., when intended), and ultimately to the accompanying end effector, that remains uninterrupted when the components rotate with respect to each other about the axis of rotation 145 (i.e., here, the shaft axis 126). The electrically conductive bearing 184 can also facilitate rotation between the first body energy hub 102 and electrically conductive shaft 172 amid roll and rotational movements about the axis of rotation 145. The electrically conductive bearing 184 is held in place at its location between the first body energy hub 102 and electrically conductive shaft 172, and establishes multiple points of contact with both components. It can rotate with the first body energy hub 102 and/or with the electrically conductive shaft 172 amid use of the handheld electrosurgical tool 12, or can remain static relative thereto. In assembly and installation, the electrically conductive bearing 184 is housed within the electrically nonconductive cover 108 and is disposed around the electrically conductive shaft 172. The electrically conductive bearing 184 makes direct surface-to-surface contact with surfaces of the first body energy hub 102. Where the electrically conductive bearing 184 is disposed over the electrically conductive shaft 172, the electrically conductive shaft 172 is free of the electrically nonconductive sleeve 174 whereby direct surface-to-surface contact is made between the shaft's electrically conductive outer surface and the electrically conductive bearing 184. The electrically conductive bearing 184 is radially sandwiched between the first body energy hub 102 and the electrically conductive shaft 172. Further, the electrically conductive bearing 184 is itself electrically conductive and can be composed of an electrically conductive material such as a metal material.

In the second embodiment, electrical energy conduction and transference spans from the external electrical energy source 142 and to the accompanying end effector by way of the rotatable energy hub assembly 18, via the electrically conductive bearing 184, and via the electrically conductive shaft 172. The flow of electrical energy transference can take the following path, per this embodiment: from the external electrical energy source 142, to and through the banana plug 100, to and through the first body energy hub 102, to and through the electrically conductive bearing 184, to and through the electrically conductive shaft 172, and ultimately to the accompanying end effector; still, the flow of electrical energy transference could involve more, less, and/or different components including intervening components in variations to this second embodiment. In one variation, for example, the flow of electrical energy transference need not involve the first body energy hub 102 which itself could be electrically nonconductive; here, the banana plug 100 could make direct contact with the electrically conductive bearing 184 and the flow of electrical energy transference would be: from the external electrical energy source 142, to and through the banana plug 100, to and through the electrically conductive bearing 184, to and through the electrically conductive shaft 172, and ultimately to the accompanying end effector. The flow of electrical energy transference can involve monopolar electrical energy, bipolar electrical energy, and/or a multitude of channels of data. Moreover, the electrical energy conduction and transference remains intact and uninterrupted amid manipulations and movements of the handle 96 and of the frame assembly 24 that are imparted to the accompanying end effector including roll and rotational movements about the axis of rotation 145.

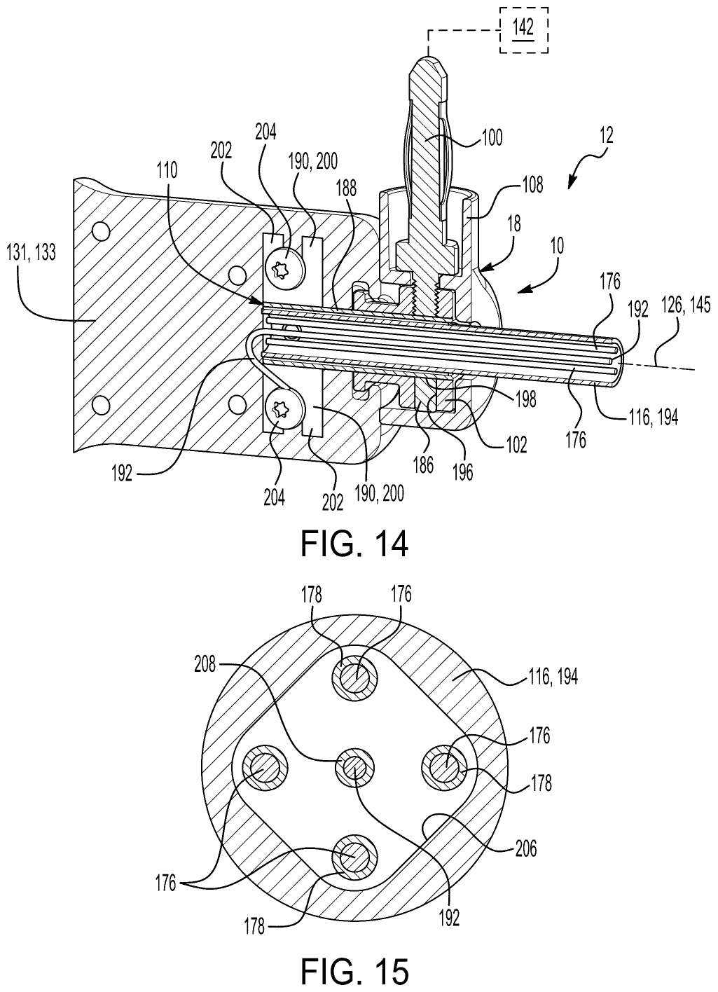

Turning now to FIGS. 14 and 15, a third embodiment of the electrical energy connection assembly 10 is presented. In the depiction and description of the third embodiment, corresponding components and elements have similar reference numerals and indications as those in previous embodiments. Moreover, similarities may exist between the previous embodiments and the third embodiment, some of which might not be repeated here in the description of the third embodiment. At least certain appreciable differences among the embodiments are set forth.

In FIGS. 14 and 15, the third embodiment has an electrically conductive pin 186 (or energy hub contact), an electrically conductive sleeve 188 (or energy sleeve), an electrically conductive holder 190 (or shaft holder, or shaft clamp, or shaft clasp), and an electrically conductive wire 192 (or energy delivery wire). Unlike the previous embodiments, the shaft 116 in the third embodiment is an electrically nonconductive shaft 194 that can be composed of an electrically nonconductive material such as a plastic material, a polymer material, a composite material, or the like. With particular reference to FIG. 14, the electrically conductive pin 186 serves to provide the continuous and maintained electrical connection between the first body energy hub 102 and electrically conductive sleeve 188 for continuous and constant electrical energy conduction and transference therebetween (i.e., when intended), and ultimately to the accompanying end effector, that remains uninterrupted when the components rotate with respect to each other about the axis of rotation 145 (i.e., here, the shaft axis 126). In this embodiment, the electrically conductive pin 186 is situated within the first body energy hub 102 and adjacent the electrically conductive sleeve 188. The electrically conductive pin 186 is carried by the first body energy hub 102 and rotates therewith amid use of the handheld electrosurgical tool 12. In assembly and installation, the electrically conductive pin 186 is housed within the electrically nonconductive cover 108. In the third embodiment, the electrically conductive pin 186 can constitute the electrical interface body 78. More specifically, a through-hole 196 receives insertion of the electrically conductive pin 186 for mounting and connection between the first body energy hub 102 and the electrically conductive pin 186. Similar to the previously-described orientation of the banana plug 100, a longitudinal axis defined by the electrically conductive pin 186 is arranged unaligned—and more particularly orthogonal—with respect to the axis of rotation 145 (i.e., here, the shaft axis 126). Furthermore, a terminal and free end portion 198 of the electrically conductive pin 186 makes and maintains direct surface-to-surface contact with an outer surface of the electrically conductive sleeve 188. The maintained contact could be via a spring load that is integral with the electrically conductive pin 186 (e.g., spring-loaded pin), or via material compliance and compression where the electrically conductive pin 186 is itself a compressible electrically conductive body; still, other ways are possible. Lastly, the electrically conductive pin 186 is itself electrically conductive and can be composed of an electrically conductive material such as a metal material.

The electrically conductive sleeve 188 extends in an axial direction between the electrically conductive pin 186 and the electrically conductive holder 190, and makes direct surface-to-surface contact with both components (axial is used here with reference to the circular and cylindrical shape of the shaft 116). The electrically conductive sleeve 188 axially overlaps with the first body energy hub 102 toward one end, and also axially overlaps with the electrically conductive holder 190 toward its other, opposite end. The electrically conductive sleeve 188-together with the first body energy hub 102 and electrically conductive pin 186-serves to provide the continuous and maintained electrical connection between the first body energy hub 102 and electrically conductive wire 192 for continuous and constant electrical energy conduction and transference therebetween (i.e., when intended), and ultimately to the accompanying end effector, that remains uninterrupted when the components rotate with respect to each other about the axis of rotation 145 (i.e., here, the shaft axis 126). In the third embodiment, the electrically conductive sleeve 188 can be a component of the conductor assembly 110 and/or can constitute the electrical interface body 78. Further, the electrically conductive sleeve 188 is rigidly attached and constrained in place to and over the electrically nonconductive shaft 194 at the shaft's proximal end, and hence rotates with the electrically nonconductive shaft 194 amid use of the handheld electrosurgical tool 12. In assembly and installation, the electrically conductive sleeve 188 is partly housed within the electrically nonconductive cover 108 and is partly housed within the shaft mount structures 131, 133 and/or the shaft mount housing. Lastly, the electrically conductive sleeve 188 is itself electrically conductive and can be composed of an electrically conductive material such as a metal material.

With continued reference to FIG. 14, the electrically conductive holder 190 serves to clamp and retain the shaft 116 (i.e., here, the electrically nonconductive shaft 194) in place and at the shaft mount structures 131, 133 and/or the shaft mount housing, and according to this embodiment serves to provide a path of electrical energy conduction and transference downstream to the electrically conductive wire 192. The electrically conductive holder 190 can have varying designs, constructions, and components in various embodiments. In the third embodiment, the electrically conductive holder 190 is in the form of a shaft clasp that, when assembled together and installed, surrounds the electrically conductive sleeve 188 and the proximal end of the electrically nonconductive shaft 194. The shaft mount structures 131, 133 and/or shaft mount housing then enclose that axial portion of the electrically conductive sleeve 188 and the shaft's proximal end and the electrically conductive holder 190. A shaft clasp structure 200 is depicted in FIG. 14. A pair of attachment projections or tabs 202 extend from a half-cylindrical structure (hidden in FIG. 14) that receives the electrically conductive sleeve 188 and the shaft's proximal end. The tabs 202 depend vertically up and down and radially relative to the electrically nonconductive shaft 194. Further, a pair of fasteners 204 are tightened down on the tabs 202 via u-shaped openings defined in the tabs 202. The fasteners 204 are in the form of screws. By way of the half-cylindrical structures of the shaft clasp, the electrically conductive holder 190 makes and maintains direct surface-to-surface contact with the outer surface of the electrically conductive sleeve 188. In this third embodiment, the electrically conductive holder 190 can be a component of the conductor assembly 110. Lastly, the electrically conductive holder 190 is itself electrically conductive and can be composed of an electrically conductive material such as a metal material.

Furthermore, in the third embodiment, the electrically conductive wire 192 serves to provide a path of electrical energy conduction and transference downstream of the electrically conductive holder 190 and ultimately to the accompanying end effector. With reference now to both FIGS. 14 and 15, the electrically conductive wire 192 extends axially from the electrically conductive holder 190 and to the end effector. It spans through the full axial extent of the electrically nonconductive shaft 194. As shown in FIG. 15, the electrically conductive wire 192 in this embodiment is routed through a central region of an interior 206 of the electrically nonconductive shaft 194. The central region can be of any shape and/or size, and can be made-up of multiple sub-regions that provide routing for one or more cables and/or wires. An electrically nonconductive wire sheath 208 is situated over an outer surface of the electrically conductive wire 192 for the purpose of electrical energy insulation. The electrically nonconductive wire sheath 208 can be composed of an electrically nonconductive material such as a plastic material. The end effector control cables 176 with the electrically nonconductive cable sheaths 178 are located peripherally around the electrically conductive wire 192 at the interior 206. In this embodiment, at the electrically conductive holder 190, a connection and associated contact is made and maintained between the electrically conductive wire 192 and the electrically conductive holder 190. Electrical energy conduction and transference occurs at the connection. Where the connection is made, the electrically conductive wire 192 is free of the electrically nonconductive wire sheath 208. The connection can take different forms in different embodiments. In the third embodiment, one of the fasteners 204 is screwed down on a terminal end of the electrically conductive wire 192 at one of the tabs 202 in order to establish the connection between the electrically conductive wire 192 and the electrically conductive holder 190. Still, in other embodiments, the connection can be via crimping, electrical connector, soldering, adhesive, epoxy, potting, and/or some other technique.

Furthermore, at the accompanying end effector, a connection and associated contact is made and maintained between the electrically conductive wire 192 and the end effector. As before, electrical energy conduction and transference occurs at the connection, and the electrically conductive wire 192 can be free of the electrically nonconductive wire sheath 208 at the site of connection. The connection at the end effector can take different forms in different embodiments including via fastening, crimping, electrical connector, soldering, adhesive, epoxy, potting, and/or some other technique. Moreover, the electrically conductive wire 192 itself can take different forms in different embodiments. It is electrically conductive and can be composed of an electrically conductive material such as a metal material. In various embodiments, the electrically conductive wire 192 can be a nitinol (i.e., nickel titanium alloy) wire, a copper wire, a braided steel cable, or something else that is capable of electrical energy conduction and transference to the end effector. In this third embodiment, the electrically conductive wire 192 can be a component of the conductor assembly 110. Here, the end effector can be an electrically conductive end effector that is composed of an electrically conductive material such as a metal material. The end effector, for example, can be a hook-shaped end effector or a hook end effector body. Still further, in other embodiments the electrically conductive wire 192 could include more than one discrete wires of the same kind or of different kinds for electrical energy transference of one or more of monopolar electrical energy, bipolar electrical energy, and/or a multitude of channels of data.

In the third embodiment, electrical energy conduction and transference spans from the external electrical energy source 142 and to the accompanying end effector by way of the rotatable energy hub assembly 18. The flow of electrical energy transference can take the following path, per this embodiment: from the external electrical energy source 142, to and through the banana plug 100, to and through the first body energy hub 102, to and through the electrically conductive pin 186, to and through the electrically conductive sleeve 188, to and through the electrically conductive holder 190, to and through the electrically conductive wire 192, and ultimately to the accompanying end effector; still, the flow of electrical energy transference could involve more, less, and/or different components including intervening components in variations to this third embodiment. The flow of electrical energy transference can involve monopolar electrical energy, bipolar electrical energy, and/or a multitude of channels of data. Moreover, the electrical energy conduction and transference remains intact and uninterrupted amid manipulations and movements of the handle 96 and of the frame assembly 24 that are imparted to the accompanying end effector including roll and rotational movements about the axis of rotation 145.

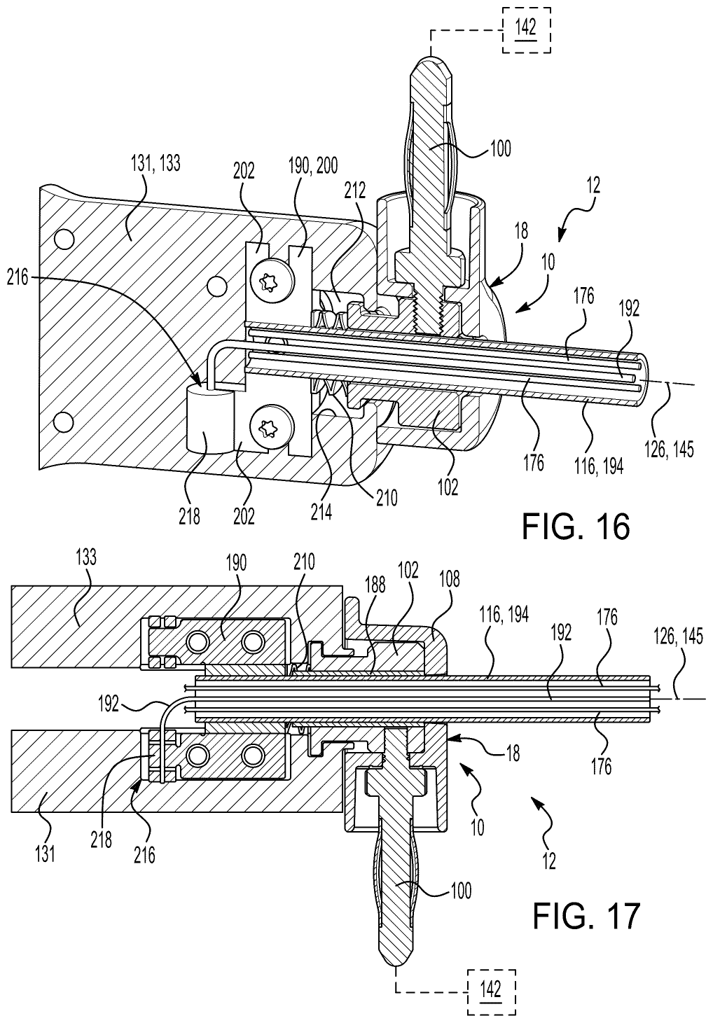

Turning now to FIG. 16, a fourth embodiment of the electrical energy connection assembly 10 is presented. In the depiction and description of the fourth embodiment, corresponding components and elements have similar reference numerals and indications as those in previous embodiments. Moreover, similarities may exist between the previous embodiments and the fourth embodiment, some of which might not be repeated here in the description of the fourth embodiment. At least certain appreciable differences among the embodiments are set forth.

In FIG. 16, the electrical interface body 78 is in the form of a wave spring 210. The wave spring 210 constitutes the electrically conductive biasing member 182 according to this embodiment. The wave spring 210 serves to provide the continuous and maintained electrical connection between the first body energy hub 102 and electrically conductive holder 190 for continuous and constant electrical energy conduction and transference therebetween (i.e., when intended), and ultimately to the accompanying end effector, that remains uninterrupted when the components rotate with respect to each other about the axis of rotation 145 (i.e., here, the shaft axis 126). In the fourth embodiment, the wave spring 210 is situated between the first body energy hub 102 and the electrically conductive holder 190, and is disposed and extends axially therebetween via a clearance 212 that otherwise spans axially therebetween. The wave spring 210 is situated circumferentially around the electrically nonconductive shaft 194 adjacent a proximal section thereof and at the shaft's outer surface, and is located within a cavity 214 defined and residing internally of the shaft mount structures 131, 133 and/or of the shaft mount housing. At its location, the wave spring 210 exerts loads against the first body energy hub 102 and against the electrically conductive holder 190, and establishes multiple points of contact with both components. Direct point-to-point and surface-to-surface contact is made between one end of the wave spring 210 and the first body energy hub 102, and is also made between an opposite end of the wave spring 210 and the electrically conductive holder 190. The wave spring 210 can rotate with the first body energy hub 102 and/or with the electrically nonconductive shaft 194 amid use of the handheld electrosurgical tool 12, or can remain static relative thereto. Further, the wave spring 210 is itself electrically conductive and can be composed of an electrically conductive material such as a metal material. Still, in alternatives to this fourth embodiment, the electrically conductive biasing member 182 could be springs of other types.

With continued reference to FIG. 16, in the fourth embodiment, the connection and associated contact made and maintained between the electrically conductive wire 192 and the electrically conductive holder 190 is in the form of a crimp connection 216. As before, electrical energy conduction and transference occurs at the crimp connection 216. In FIG. 16, an extension 218 of the electrically conductive holder 190 (and of one of the tabs 202) is deformed over and squeezed down on a terminal end of the electrically conductive wire 192 in order to establish the connection therebetween. A crimping tool may be employed for this purpose. Where the crimp connection 216 is effected, the terminal end of the electrically conductive wire 192 is free of the electrically nonconductive wire sheath 208. Still, in alternative embodiments, this connection can be via screwing, electrical connector, soldering, adhesive, epoxy, potting, and/or some other technique.

In the fourth embodiment, electrical energy conduction and transference spans from the external electrical energy source 142 and to the accompanying end effector by way of the rotatable energy hub assembly 18, via the wave spring 210, and via the electrically conductive wire 192. The flow of electrical energy transference can take the following path, per this embodiment: from the external electrical energy source 142, to and through the banana plug 100, to and through the first body energy hub 102, to and through the wave spring 210, to and through the electrically conductive holder 190, to and through the electrically conductive wire 192, and ultimately to the accompanying end effector; still, the flow of electrical energy transference could involve more, less, and/or different components including intervening components in variations to this fourth embodiment. The flow of electrical energy transference can involve monopolar electrical energy, bipolar electrical energy, and/or a multitude of channels of data. Moreover, the electrical energy conduction and transference remains intact and uninterrupted amid manipulations and movements of the handle 96 and of the frame assembly 24 that are imparted to the accompanying end effector including roll and rotational movements about the axis of rotation 145.

Turning now to FIG. 17, a fifth embodiment of the electrical energy connection assembly 10 is presented. In the depiction and description of the fifth embodiment, corresponding components and elements have similar reference numerals and indications as those in previous embodiments. Moreover, similarities may exist between the previous embodiments and the fifth embodiment, some of which might not be repeated here in the description of the fifth embodiment. At least certain appreciable differences among the embodiments are set forth.

The fifth embodiment shares many designs, constructions, and components with the fourth embodiment. One appreciable difference is the addition of the electrically conductive sleeve 188 in this fifth embodiment. Here, the electrically conductive sleeve 188 extends in the axial direction between the first body energy hub 102 and the wave spring 210, and makes direct surface-to-surface contact with both components. The electrically conductive sleeve 188 axially overlaps with the first body energy hub 102 toward one end, and also axially overlaps with the wave spring 210 toward its other, opposite end. The electrically conductive sleeve 188-together with the wave spring 210-serves to facilitate the provision of the continuous and maintained electrical connection between the first body energy hub 102 and electrically conductive holder 190 for continuous and constant electrical energy conduction and transference therebetween (i.e., when intended), and ultimately to the accompanying end effector that remains uninterrupted when the components rotate with respect to each other about the axis of rotation 145 (i.e., here, the shaft axis 126). In the fifth embodiment, the electrically conductive sleeve 188 can be a component of the conductor assembly 110 and/or can constitute the electrical interface body 78. Further, the electrically conductive sleeve 188 is rigidly attached and constrained in place to and over the electrically nonconductive shaft 194 at the shaft's proximal end, and hence rotates with the electrically nonconductive shaft 194 amid use of the handheld electrosurgical tool 12. In assembly and installation, the electrically conductive sleeve 188 is partly housed within the electrically nonconductive cover 108 and is partly housed within the shaft mount structures 131, 133 and/or the shaft mount housing. Lastly, the electrically conductive sleeve 188 is itself electrically conductive and can be composed of an electrically conductive material such as a metal material.

In the fifth embodiment, electrical energy conduction and transference spans from the external electrical energy source 142 and to the accompanying end effector. The flow of electrical energy transference can take the following path, per this embodiment: from the external electrical energy source 142, to and through the banana plug 100, to and through the first body energy hub 102, to and through the electrically conductive sleeve 188, to and through the wave spring 210, to and through the electrically conductive holder 190, to and through the electrically conductive wire 192, and ultimately to the accompanying end effector; still, the flow of electrical energy transference could involve more, less, and/or different components including intervening components in variations to this fifth embodiment. The flow of electrical energy transference can involve monopolar electrical energy, bipolar electrical energy, and/or a multitude of channels of data. Moreover, the electrical energy conduction and transference remains intact and uninterrupted amid manipulations and movements of the handle 96 and of the frame assembly 24 that are imparted to the accompanying end effector including roll and rotational movements about the axis of rotation 145.

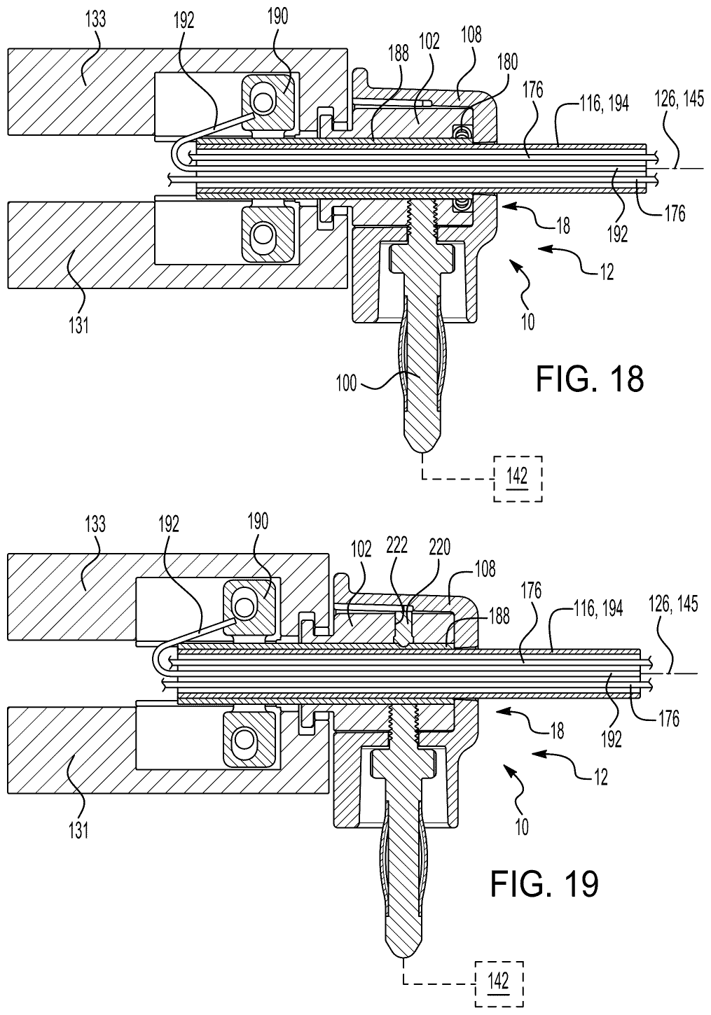

Turning now to FIG. 18, a sixth embodiment of the electrical energy connection assembly 10 is presented. In the depiction and description of the sixth embodiment, corresponding components and elements have similar reference numerals and indications as those in previous embodiments. Moreover, similarities may exist between the previous embodiments and the sixth embodiment, some of which might not be repeated here in the description of the sixth embodiment. At least certain appreciable differences among the embodiments are set forth.

The sixth embodiment shares many designs, constructions, and components with the first and third embodiments. In the sixth embodiment, the electrically conductive sleeve 188 extends in the axial direction between the canted coil spring 180 and the electrically conductive holder 190, and makes direct surface-to-surface contact with both components. The electrically conductive sleeve 188 axially overlaps with the canted coil spring 180 and the first body energy hub 102 toward one end, and axially overlaps with the electrically conductive holder 190 toward its other, opposite end. The electrically conductive sleeve 188-together with the canted coil spring 180-serves to facilitate the provision of the continuous and maintained electrical connection between the first body energy hub 102 and electrically conductive holder 190 for continuous and constant electrical energy conduction and transference therebetween (i.e., when intended), and ultimately to the accompanying end effector that remains uninterrupted when the components rotate with respect to each other about the axis of rotation 145 (i.e., here, the shaft axis 126). In the sixth embodiment, the electrically conductive sleeve 188 can be a component of the conductor assembly 110 and/or can constitute the electrical interface body 78. Further, the electrically conductive sleeve 188 is rigidly attached and constrained in place to and over the electrically nonconductive shaft 194 at the shaft's proximal end, and hence rotates with the electrically nonconductive shaft 194 amid use of the handheld electrosurgical tool 12. Furthermore, the canted coil spring 180 is trapped in place at its location between the first body energy hub 102 and electrically conductive sleeve 188, and establishes multiple points of contact with both components and exerts loads therebetween. It can rotate with the first body energy hub 102 and/or with the electrically conductive sleeve 188 amid use of the handheld electrosurgical tool 12, or can remain static relative thereto. In the sixth embodiment, the canted coil spring 180 is the electrical interface body 78 and constitutes an electrically conductive biasing member 182. In assembly and installation, the canted coil spring 180 is disposed circumferentially around the electrically conductive sleeve 188. The canted coil spring 180 is radially sandwiched between the first body energy hub 102 and the electrically conductive sleeve 188.

In the sixth embodiment, electrical energy conduction and transference spans from the external electrical energy source 142 and to the accompanying end effector. The flow of electrical energy transference can take the following path, per this embodiment: from the external electrical energy source 142, to and through the banana plug 100, to and through the first body energy hub 102, to and through the canted coil spring 180, to and through the electrically conductive sleeve 188, to and through the electrically conductive holder 190, to and through the electrically conductive wire 192, and ultimately to the accompanying end effector; still, the flow of electrical energy transference could involve more, less, and/or different components including intervening components in variations to this sixth embodiment. The flow of electrical energy transference can involve monopolar electrical energy, bipolar electrical energy, and/or a multitude of channels of data. Moreover, the electrical energy conduction and transference remains intact and uninterrupted amid manipulations and movements of the handle 96 and of the frame assembly 24 that are imparted to the accompanying end effector including roll and rotational movements about the axis of rotation 145.

Turning now to FIG. 19, a seventh embodiment of the electrical energy connection assembly 10 is presented. In the depiction and description of the seventh embodiment, corresponding components and elements have similar reference numerals and indications as those in previous embodiments. Moreover, similarities may exist between the previous embodiments and the seventh embodiment, some of which might not be repeated here in the description of the seventh embodiment. At least certain appreciable differences among the embodiments are set forth.

The seventh embodiment shares many designs, constructions, and components with the third and sixth embodiments. In the seventh embodiment, the electrically conductive sleeve 188 extends in the axial direction between an electrically conductive pin 220 and the electrically conductive holder 190, and makes direct surface-to-surface contact with both components. The electrically conductive sleeve 188 axially overlaps with the electrically conductive pin 220 and the first body energy hub 102 toward one end, and axially overlaps with the electrically conductive holder 190 toward its other, opposite end. The electrically conductive sleeve 188-together with the electrically conductive pin 220-serves to facilitate the provision of the continuous and maintained electrical connection between the first body energy hub 102 and electrically conductive holder 190 for continuous and constant electrical energy conduction and transference therebetween (i.e., when intended), and ultimately to the accompanying end effector that remains uninterrupted when the components rotate with respect to each other about the axis of rotation 145 (i.e., here, the shaft axis 126). In the seventh embodiment, the electrically conductive sleeve 188 can be a component of the conductor assembly 110 and/or can constitute the electrical interface body 78. Further, the electrically conductive sleeve 188 is rigidly attached and constrained in place to and over the electrically nonconductive shaft 194 at the shaft's proximal end, and hence rotates with the electrically nonconductive shaft 194 amid use of the handheld electrosurgical tool 12. Furthermore, the electrically conductive pin 220 is situated within the first body energy hub 102 and adjacent the electrically conductive sleeve 188. The electrically conductive pin 220 is carried by the first body energy hub 102 and rotates therewith amid use of the handheld electrosurgical tool 12. In assembly and installation, the electrically conductive pin 220 is housed within the electrically nonconductive cover 108. In the seventh embodiment, the electrically conductive pin 220 can constitute the electrical interface body 78. More specifically, a through-hole 222 receives insertion of the electrically conductive pin 220 for mounting and connection between the first body energy hub 102 and the electrically conductive pin 220. Here, the electrically conductive pin 220 is in the form of a pogo or spring-loaded pin having a moveable plunger, barrel, and internal helical spring. A terminal and free end portion of the electrically conductive pin 220 makes and maintains direct surface-to-surface point contact with an outer surface of the electrically conductive sleeve 188, and exerts loads thereto. Lastly, the electrically conductive pin 220 is itself electrically conductive and can be composed of an electrically conductive material such as a metal material.

In the seventh embodiment, electrical energy conduction and transference spans from the external electrical energy source 142 and to the accompanying end effector. The flow of electrical energy transference can take the following path, per this embodiment: from the external electrical energy source 142, to and through the banana plug 100, to and through the first body energy hub 102, to and through the electrically conductive pin 220, to and through the electrically conductive sleeve 188, to and through the electrically conductive holder 190, to and through the electrically conductive wire 192, and ultimately to the accompanying end effector; still, the flow of electrical energy transference could involve more, less, and/or different components including intervening components in variations to this seventh embodiment. The flow of electrical energy transference can involve monopolar electrical energy, bipolar electrical energy, and/or a multitude of channels of data. Moreover, the electrical energy conduction and transference remains intact and uninterrupted amid manipulations and movements of the handle 96 and of the frame assembly 24 that are imparted to the accompanying end effector including roll and rotational movements about the axis of rotation 145.

As described, it has been found that certain embodiments of the electrical interface body 78 are mechanically compliant bodies and serve to accommodate potential variances and imprecisions of dimensions introduced by adjacent components and bodies. The canted coil spring 180, wave spring 210, and electrically conductive pin 220 serve as examples that exhibit such accommodation. The accommodation has been shown to facilitate the provision of substantially continuous and maintained electrical connection (i.e., when intended) among the components that remains substantially uninterrupted when the components rotate with respect to each other.

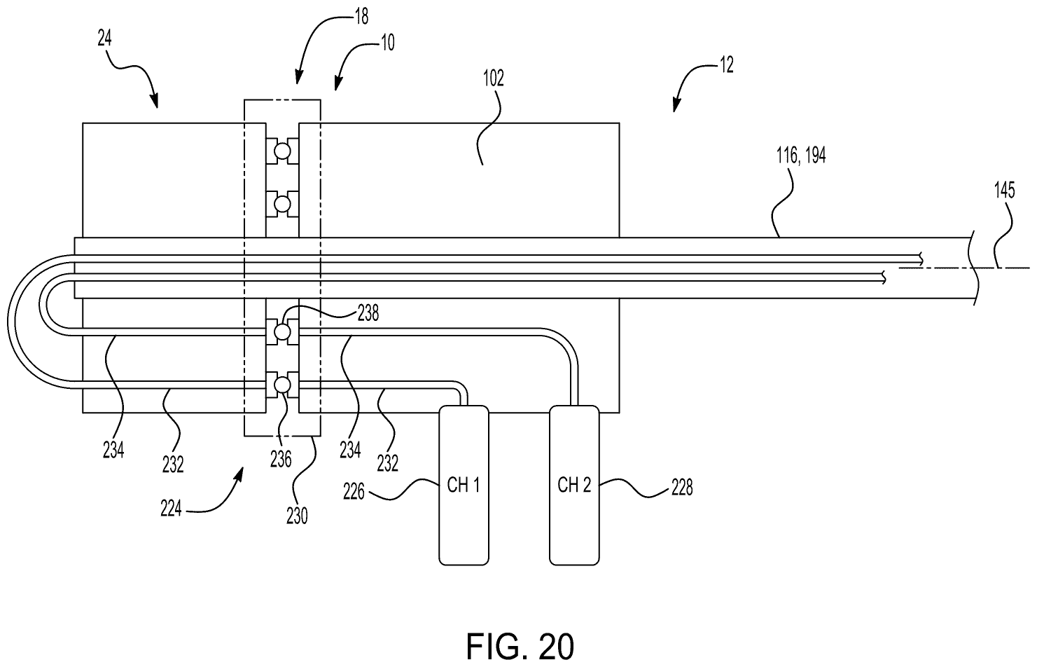

Turning now to FIG. 20, an eighth embodiment of the electrical energy connection assembly 10 is presented. In the depiction and description of the eighth embodiment, corresponding components and elements have similar reference numerals and indications as those in previous embodiments. Moreover, similarities may exist between the previous embodiments and the eighth embodiment, some of which might not be repeated here in the description of the eighth embodiment. At least certain appreciable differences among the embodiments are set forth.

In the eighth embodiment, the electrical energy connection assembly 10 involves a bipolar electrical energy transference assembly 224. Bipolar electrical energy transference is facilitated at the electrical energy connection assembly 10 via the bipolar electrical energy transference assembly 224. The bipolar electrical energy transference assembly 224 serves to facilitate the provision of the continuous and maintained electrical connection between the rotatable energy hub assembly 18 and the frame assembly 24 for continuous and constant electrical energy conduction and transference (i.e., when intended) therebetween (i.e., here, bipolar electrical energy conduction and transference), and ultimately to the accompanying end effector that remains uninterrupted when the components rotate with respect to each other about the axis of rotation 145. The bipolar electrical energy transference assembly 224 can have varying designs, constructions, and components in various embodiments. In FIG. 20, the bipolar electrical energy transference assembly 224 has a first channel 226, a second channel 228, and a slip ring joint assembly 230 (denoted by broken line rectangle; still, the bipolar electrical energy transference assembly could involve more, less, and/or different components including intervening components in variations to this eighth embodiment.

The first and second channels 226, 228 serve as electrical energy conductor paths in the bipolar electrical energy transference assembly 224. Here, the first channel 226 constitutes an electrical energy supply path, and the second channel 228 constitutes an electrical energy return path. In application, the first and second channels 226, 228 can be connected to the external electrical energy source 142 which may supply bipolar electrical energy. In this embodiment, first and second electrically conductive wires 232, 234 are provided and span through the first body energy hub 102, through components and/or portions of the frame assembly 24, and through the interface therebetween. Further, the first and second electrically conductive wires 232, 234 span through, and are routed through, the axial extent of the electrically nonconductive shaft 194 and to the accompanying end effector. The first and second electrically conductive wires 232, 234 exhibit continuous and maintained electrical connection via the slip ring joint assembly 230 for continuous and constant bipolar electrical energy conduction and transference (i.e., when intended) that remains uninterrupted when rotation occurs between the respective components at the slip ring joint assembly 230 and about the axis of rotation 145. The electrical connection remains intact over all relative rotational movements and positions between the first body energy hub 102 and the frame assembly 24, per this embodiment. Furthermore, as represented by their separate locations in FIG. 20, the first and second channels 226, 228 and first and second electrically conductive wires 232, 234 can be spaced and distanced apart from each other in order to preclude unintended electrical energy and/or signal leakage therebetween.

The slip ring joint assembly 230—also known as a rotary electrical interface—serves to enable electrical energy conduction and transference amid relative rotations of one of the components interfaced by the slip ring joint assembly 230 (here, the first body energy hub 102 or the frame assembly 24) with respect to the other. Electrical energy conduction and transference remains at first and second rotational interfaces 236, 238 of the first and second electrically conductive wires 232, 234 via the slip ring joint assembly 230. The slip ring joint assembly 230 can take varying forms in various implementations, dictated at least in part by the intended magnitude of electrical energy conduction and transference to occur and/or the extent of rotations to be endured at the slip ring joint assembly 230, among other potential factors. Moreover, in variations to this eighth embodiment, the slip ring joint assembly 230 can be designed and constructed to enable a multiplicity of electrical energy channels and/or wires and the accompanying electrical energy conduction and transference at the slip ring joint assembly 230. For example, the slip ring joint assembly 230 could involve numerous channels of data being exchanged. For such scalability, at least per one variation of the eighth embodiment, an encoder disk could be utilized. The encoder disk could have multiple sectors with each sector constituting a discrete channel of data. The sectors could be positioned radially relative to one another. As a further variation to the eighth embodiment, the electrical energy connection assembly 10 could be designed and constructed for implementation in a motorized handheld tool application and in a medical device application or a non-medical device application. Here, a multiplicity of electrical energy channels and/or wires could be provided, including a multiplicity of control channels and/or wires. In such a variation, one or more motors could be equipped adjacent the electrical energy connection assembly 10, and could be user activated and deactivated for control and operation of end effectors for opening and closing, rotating, cutting, cauterizing, grasping, holding, dissecting, suturing, and/or clamping, among other potential functionalities taking place via the end effectors.

Furthermore, in general, while a multitude of embodiments have been depicted and described with a multitude of components in each embodiment, in alternative embodiments of the electrical energy connection assembly the components of various embodiments—e.g., those of the first, second, and/or third embodiments-could be intermixed, combined, and/or exchanged for one another. In other words, components described in connection with a particular embodiment are not necessarily exclusive to that particular embodiment.

As used herein, the terms “general” and “generally” and “substantially” are intended to account for the inherent degree of variance and imprecision that is often attributed to, and often accompanies, any design and manufacturing process, including engineering tolerances—and without deviation from the relevant functionality and intended outcome-such that mathematical precision and exactitude is not implied and, in some instances, is not possible. In other instances, the terms “general” and “generally” and “substantially” are intended to represent the inherent degree of uncertainty that is often attributed to any quantitative comparison, value, and measurement calculation, or other representation.

It is to be understood that the foregoing is a description of one or more aspects of the disclosure. The disclosure is not limited to the particular embodiment(s) disclosed herein, but rather is defined solely by the claims below. Furthermore, the statements contained in the foregoing description relate to particular embodiments and are not to be construed as limitations on the scope of the disclosure or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “e.g.,” “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

Those of skill in the art will understand that modifications (additions and/or removals) of various components of the substances, formulations, apparatuses, methods, systems, and embodiments described herein may be made without departing from the full scope and spirit of the present disclosure, which encompass such modifications and any and all equivalents thereof.

1. An electrical energy connection assembly, comprising:
a user input assembly having at least one electrically nonconductive body;
a first electrically conductive body constrained translationally to said at least one electrically nonconductive body and having a rotational degree of freedom with respect to said at least one electrically nonconductive body and about an axis of rotation of said at least one electrically nonconductive body, said rotational degree of freedom providing full rotational capabilities of said first electrically conductive body and said at least one electrically nonconductive body with respect to each other;
at least one electrical connector carried by said first electrically conductive body and rotatable therewith about said rotational degree of freedom; and
at least one second electrically conductive body;
wherein, when said at least one electrical connector is connected to an external electrical energy source, electrical energy is conducted from said at least one electrical connector, to said first electrically conductive body, to said at least one second electrically conductive body, and to an output body located remote of said user input assembly.

2. The electrical energy connection assembly as set forth in claim 1, further comprising an electrically nonconductive housing at least partially surrounding said first electrically conductive body and at least partially surrounding said at least one electrical connector.

3. The electrical energy connection assembly as set forth in claim 1, wherein said at least one second electrically conductive body includes an electrically conductive biasing member situated adjacent said first electrically conductive body and substantially maintaining contact with said first electrically conductive body and with another electrically conductive body of said at least one second electrically conductive body amid rotational movements of said first electrically conductive body and said at least one electrically nonconductive body with respect to each other.

4. The electrical energy connection assembly as set forth in claim 3, wherein said at least one second electrically conductive body further includes an electrically conductive sleeve situated adjacent said electrically conductive biasing member, said electrically conductive biasing member substantially maintaining contact with said electrically conductive sleeve for facilitation of electrical energy conduction therebetween.

5. The electrical energy connection assembly as set forth in claim 1, wherein said at least one second electrically conductive body includes an electrically conductive bearing situated adjacent said first electrically conductive body and substantially maintaining contact with said first electrically conductive body and with another electrically conductive body of said at least one second electrically conductive body amid rotational movements of said first electrically conductive body and said at least one electrically nonconductive body with respect to each other.

6. The electrical energy connection assembly as set forth in claim 1, wherein said at least one second electrically conductive body includes an electrically conductive biasing member and an electrically conductive holder, said electrically conductive biasing member situated adjacent said first electrically conductive body and said electrically conductive holder and substantially maintaining contact with said first electrically conductive body and with said electrically conductive holder amid rotational movements of said first electrically conductive body and said at least one electrically nonconductive body with respect to each other.

7. The electrical energy connection assembly as set forth in claim 6, wherein said electrically conductive biasing member is an electrically conductive wave spring.

8. The electrical energy connection assembly as set forth in claim 6, wherein said at least one second electrically conductive body further includes a shaft, said electrically conductive holder retaining said shaft, said shaft extending from said user input assembly and to the output body.

9. The electrical energy connection assembly as set forth in claim 8, wherein said at least one second body further includes an electrically conductive wire, said electrically conductive wire making contact with said electrically conductive holder and extending to the output body.

10. The electrical energy connection assembly as set forth in claim 1, wherein said at least one second electrically conductive body includes an electrically conductive pin situated adjacent said first electrically conductive body and substantially maintaining contact with said first electrically conductive body and with another electrically conductive body of said at least one second electrically conductive body amid rotational movements of said first electrically conductive body and said at least one electrically nonconductive body with respect to each other.

11. The electrical energy connection assembly as set forth in claim 10, wherein said at least one second electrically conductive body further includes an electrically conductive sleeve situated adjacent said electrically conductive pin, said electrically conductive pin substantially maintaining contact with said electrically conductive sleeve for facilitation of electrical energy conduction therebetween.

12. The electrical energy connection assembly as set forth in claim 1, wherein said first electrically conductive body is an electrically conductive hub body.

13. The electrical energy connection assembly as set forth in claim 1, wherein the electrical energy connection assembly facilitates monopolar electrical energy transference via said at least one electrical connector.

14. The electrical energy connection assembly as set forth in claim 1, wherein the electrical energy connection assembly facilitates bipolar electrical energy transference via said at least one electrical connector.

15. The electrical energy connection assembly as set forth in claim 1, wherein said at least one electrical connector has a longitudinal axis, said longitudinal axis exhibiting an unaligned arrangement with respect to said axis of rotation of said at least one electrically nonconductive body.

16. The electrical energy connection assembly as set forth in claim 15, wherein said longitudinal axis has a perpendicular arrangement with respect to said axis of rotation of said at least one electrically nonconductive body.

17. The electrical energy connection assembly as set forth in claim 1, wherein movement of said user input assembly is transmitted to the output body.

18. The electrical energy connection assembly as set forth in claim 1, wherein said at least one second body includes an electrically conductive holder and a shaft, said electrically conductive holder retaining said shaft, said shaft extending from said user input assembly and to the output body;
wherein, when said electrical connector is connected to the external electrical energy source, electrical energy is conducted from said at least one electrical connector, to said first electrically conductive body, to said at least one second electrically conductive body, to said electrically conductive holder, and to the output body.

19. The electrical energy connection assembly as set forth in claim 18, wherein said shaft is an electrically conductive shaft.

20. The electrical energy connection assembly as set forth in claim 18, wherein said at least one second body further includes an electrically conductive wire, said electrically conductive wire making contact with said electrically conductive holder and extending to the output body.

21. The electrical energy connection assembly as set forth in claim 1, further comprising a shaft extending from said user input assembly and extending to the output body, and wherein said axis of rotation is of said shaft.

22. The electrical energy connection assembly as set forth in claim 1, wherein the full rotational capabilities provided by said rotational degree of freedom provides unlimited rotational capabilities of said electrically conductive body and said electrically nonconductive body with respect to each other.

23. The electrical energy connection assembly as set forth in claim 22, wherein the electrical energy conduction from said at least one electrical connector and to said first electrically conductive body and to said at least one second electrically conductive body remains substantially uninterrupted amid unlimited rotations between said first electrically conductive body and said at least one electrically nonconductive body about said rotational degree of freedom.

24. The electrical energy connection assembly as set forth in claim 1, wherein the electrical energy connection assembly is a handheld surgical instrument electrical energy connection assembly.

25. The electrical energy connection assembly as set forth in claim 1, further comprising the output body, and wherein the output body is a medical device end effector assembly.

26. The electrical energy connection assembly as set forth in claim 1, wherein said user input assembly and said at least one electrically nonconductive body transmit roll functionality to the output body.

27. The electrical energy connection assembly as set forth in claim 1, further comprising an end effector assembly, said end effector assembly comprising a shaft body, an electrically conductive wire, and an electrically conductive end effector body, said electrically conductive wire extending at least partly through said shaft body, said electrically conductive end effector body having a connection with said electrically conductive wire, and wherein electrical energy is deliverable from said electrically conductive wire to said electrically conductive end effector body via said connection.

28. The electrical energy connection assembly as set forth in claim 27, wherein said electrically conductive end effector body is an electrically conductive end effector hook body, and said connection is a crimp connection.

29. An electrical energy connection assembly, comprising:
a first electrically conductive body; and
a second electrically conductive body constrained translationally with respect to said first electrically conductive body and having a rotational degree of freedom with respect to said first electrically conductive body, said rotational degree of freedom providing unlimited rotational capabilities of said first electrically conductive body and said second electrically conductive body with respect to each other;
wherein, when the electrical energy connection assembly is connected to an external electrical energy source, electrical energy is conducted from said first electrically conductive body, to said second electrically conductive body, and to an output body located remote of said first electrically conductive body and remote of said second electrically conductive body, and wherein the electrical energy conduction remains substantially uninterrupted amid unlimited rotations between said first electrically conductive body and said second electrically conductive body.

30. A method of delivering electrical energy between electrically conductive bodies, the method comprising:
providing a translational degree of constraint between a first electrically conductive body and a second electrically conductive body, and providing an unlimited rotational degree of freedom between said first electrically conductive body and said second electrically conductive body;
wherein, upon connection to an external electrical energy source, electrical energy is delivered from said first electrically conductive body and to said second electrically conductive body, and the electrical energy delivery between said first and second electrically conductive bodies is substantially uninterrupted amid unlimited rotations between said first and second electrically conductive bodies about said unlimited rotational degree of freedom.

31. The method of delivering electrical energy between electrically conductive bodies as set forth in claim 30, further comprising providing monopolar electrical energy transference between said first and second electrically conductive bodies via at least one electrical connector upon connection to the external electrical energy source.

32. The method of delivering electrical energy between electrically conductive bodies as set forth in claim 30, further comprising providing bipolar electrical energy transference between said first and second electrically conductive bodies via at least one electrical connector upon connection to the external electrical energy source.

33. The method of delivering electrical energy between electrically conductive bodies as set forth in claim 30, further comprising maintaining contact between said first and second electrically conductive bodies for effecting substantially uninterrupted electrical energy delivery between said first and second electrically conductive bodies amid unlimited rotations between said first and second electrically conductive bodies.
